(19) United States
(12) Patent Application Publication
(10) Pub. No.: US 2008/0020386 A1
(43) Pub. Date: Jan. 24, 2008

(54) METHODS AND APPARATUS FOR GENOTYPING

(75) Inventors: Chaw Yuan Michael Chen, Taipei City (TW); Yen-Chin Chen, Taipei City (TW); Wei-Ying Kuo, Taipei City (TW); Chiao-Chien Hung, Taipei City (TW)

Correspondence Address: DINSMORE & SHOHL LLP, ONE DAYTON CENTRE, ONE SOUTH MAIN STREET, SUITE 1300, DAYTON, OH, 45402-2023, US

(73) Assignee: MEDIGEN BIOTECHNOLOGY CORPORATION, Taipei City (TW)

(21) Appl. No.: 11/692,565

(22) Filed: Mar. 28, 2007

Related U.S. Application Data
(60) Provisional application No. 60/743,992, filed on Mar. 30, 2006.

(30) Foreign Application Priority Data
Mar. 29, 2006 (GB) GB 0606297.0

(51) Int. Cl. C12Q 1/68; C40B 40/06; G06F 19/00

(52) U.S. Cl. 435/6; 506/16; 702/20

(57) ABSTRACT

A method for determining the human leukocyte antigen (HLA) genotype of a nucleic acid sample, comprises: contacting a nucleic acid sample with at least one nucleic acid primer set and subjecting the mixture to a nucleic acid amplification reaction; determining the size of any amplification products produced in the amplification reaction; and correlating the presence and/or absence of specific amplification products with the presence and/or absence of specific sequence polymorphisms in the nucleic acid sample. At least one of the primer sets is a multi-specific primer set comprising at least one sequence-specific forward primer and at least one sequence-specific reverse primer and being adapted to amplify two or more specific target sequences in the nucleic acid sample. Each of the specific target sequences comprises a sequence polymorphism that is known to be associated with an HLA allele and which may be present in the nucleic acid sample to be genotyped.

### CROSS-REFERENCE TO RELATED APPLICATIONS

This application claims the benefit of British application GB 0606297.0 filed Mar. 29, 2006 and U.S. Provisional Application Ser. No. 60/743,992 filed Mar. 30, 2006.

### FIELD OF THE INVENTION

The invention relates to methods and apparatus for determining the genotype of a sample of genetic material derived from a test subject. More specifically, the invention relates to sequence-specific primer PCR (PCR-SSP) genotyping of the major histocompatibility complex (MHC), such as human leukocyte antigen (HLA) typing.

### BACKGROUND OF THE INVENTION

Major histocompatibility (MHC) antigens are key elements in restricting the specificity of T-cell mediated immune responses. Class I MHC molecules are expressed at the cell surface of most nucleated cells and present the peptides derived from the processing of intracellular proteins to CD8⁺ T-cells. Class II MHC molecules are expressed at the cell surface of antigen presenting cells (APCs, for example, macrophages and B-cells) and present to CD4⁺ T cells antigens derived from the processing of phagocytosed extracellular antigens.

Human MHC molecules were initially discovered following rejection of skin grafts used to treat burns victims during World War II. It was soon appreciated that the rejection was mediated by an immune reaction against MHC molecules of the donor expressed on the cells of the transplanted skin. Experiments revealed that these molecules were encoded, and their expression controlled, by a single genetic region: due to its role in determining compatibility of tissue transplants, the region was termed the major histocompatibility complex (MHC). In humans, the MHC genes are known as the human leukocyte antigen (HLA) genes; in pigs, as the swine leukocyte antigen (SLA) genes; and in mice, as the H-2 genes. Classical HLA class I genes include HLA-A, HLA-B and HLA-C. Classical HLA class II genes include pairs of HLA-DP, HLA-DQ and HLA-DR.

MHC genes are highly polymorphic, and are, in fact, the most polymorphic genes known. Thus, expression of the HLA genes can yield MHC molecules that differ distinctly in sequence between individuals of the same species. In addition, as each individual expresses three types of MHC class I molecules (products of the polymorphic HLA-A, HLA-B and HLA-C genes) and typically three or four types of MHC class II molecules (dimeric products of the polymorphic HLA-DP, HLA-DQ and HLA-DR gene pairs), the HLA profile of each individual is highly specific.

Transplantation of bone marrow, organs and tissues between individuals is now an important medical therapy; transplants of this kind are known as allografts. Unfortunately, unless the donor and recipient individuals are genetically identical (e.g. identical twins), rejection of transplanted tissue is almost certain. Rejection is mediated by specific T-cell responses to the MHC (in humans—HLA) molecules on the surface of the cells of the foreign tissue, which are recognised as non-self.

Accurate determination of allelic subtypes is essential for typing potential transplantation donors, where very precise HLA matching is critical in minimising risk of rejection and graft-versus-host disease (in which lymphocytes from the graft recognise the host tissue MHC molecules as non-self and mount an immune response against the host). Recent studies also suggest the association between HLA alleles and several diseases, including type 1 diabetes mellitus, and ankylosing spondylitis.

The limited availability of tissue donors and the short amount of time available to identify a suitable recipient when a donor organ becomes available, mean that HLA typing must be done as quickly and accurately as possible. However, current HLA typing methods are limited and the success of solid organ transplantation is more the result of post-operative administration of immunosuppressive drugs, rather than of accurate pre-operative tissue typing (Janeway et al., 2005. Autoimmunity and transplantation, in Immunobiology: The Immune System in Health and Disease. Garland Science Publishing, New York, N.Y.). The urgent need to perform HLA typing quickly and accurately places considerable pressure upon the technical staff who are required to perform such typing, particularly as typing may need to be done at unsociable hours and with little advanced warning. Hence, the introduction of human error into HLA typing reactions is a real and unavoidable consequence of this pressure.

Traditionally, HLA typing for tissue matching has been performed by serological and cellular methods. For example, monoclonal antibodies specific to HLA antigens can be added to white blood cells, together with complement and dye; if the white blood cells express the MHC allele detected by the particular monoclonal antibody, then the cells will be lysed upon addition of complement and the dead cells will take up the dye. However, major problems of serological typing arise from the requirements for live cells and HLA allele-specific monoclonal antibodies.

Several PCR-based methods for HLA typing have also been developed, such as PCR-RFLP (Restriction Fragment Length Polymorphism), PCR-SSO (Sequence-Specific Oligonucleotide probe), PCR-SSCP (Single Strand Conformation Polymorphism) and PCR-SSP (Sequence-Specific Primers). In each of these methods the gene region to be analysed is amplified by the PCR method and, typically, the variable region in the sequences of the amplified products is then analysed by combination with other resolution techniques in order to distinguish the genotype.

In PCR-RFLP, the amplified products are subject to restriction endonuclease digestion, and the digested products are separated according to their size using electrophoresis. Determination of the genotype depends on the presence or absence of fragments of certain sizes. This method is often considered as overly time-consuming for clinical use. Furthermore, RFLP does not generally detect polymorphisms within the exons, but relies upon the strong linkage between allele-specific nucleotide sequences and restriction endonuclease recognition sites within surrounding region, generally in non-coding regions such as introns.

In PCR-SSO, genotyping depends on the hybridisation of the amplified products with sequence-specific oligonucleotide probes. Typically, one of the amplified products or probes is labelled, and one of the probes or amplified products is immobilised on a substrate. For example, if the probes are immobilised, the amplified products are labelled, and vice versa. After the hybridisation and washing steps, detection of hybridisation is performed in order to determine the genotype. This method, which requires several steps of manipulation, is relatively complex and time consuming.

In PCR-SSCP, the PCR products are made single stranded and the single stranded products of specific regions are separated using non-denaturing polyacrylamide gel electrophoresis. Single strands with different nucleotide compositions migrate at different speeds. By comparison with known standards, the genotype can be assigned. Therefore, many controls have to be included to determine a viable genotype. This method is extremely time consuming and labour intensive.

In PCR-SSP, allelic sequence-specific primers are designed to amplify only the specified allele. Detection of the amplification products is usually done by agarose gel electrophoresis followed by ethidium bromide (EtBr) staining of the gel. Determination of the genotype depends on the presence or absence of the appropriate amplification product. This method requires the design of a large number of sequence-specific PCR primers and the operator to handle a large number of PCR reactions. The electrophoresis process can also take a long time, and using standard slab gel electrophoresis is not easily adapted for automation.

Accordingly, it is the object of the present invention to improve the accuracy and speed of MHC genotyping, and also to reduce the human error inherent in the performance of MHC genotyping, particularly HLA genotyping.

These and other uses, features and advantages of the invention should be apparent to those skilled in the art from the teachings provided herein.

### SUMMARY OF THE INVENTION

In a first aspect of the invention, there is provided an in vitro method for determining the human leukocyte antigen (HLA) genotype of a nucleic acid sample, comprising the steps of: providing at least one oligonucleotide primer set; contacting the nucleic acid sample with the at least one primer set and subjecting the nucleic acid sample and at least one primer set to a nucleic acid amplification reaction; determining the size of any nucleic acid amplification products produced in the nucleic acid amplification reaction; and correlating the presence and/or absence of specific amplification products with the presence and/or absence of specific sequence polymorphisms in the nucleic acid sample; wherein at least one of the at least one primer sets is a multi-specific primer set comprising at least one sequence-specific forward primer and at least one sequence-specific reverse primer and being adapted to amplify, in a nucleic acid amplification reaction, two or more specific target sequences that may be present in the nucleic acid sample, and wherein each of the specific target sequences comprises a sequence polymorphism that is known to be associated with an HLA allele and which may be present in the nucleic acid sample to be genotyped.

A preferred means of resolving the products of a nucleic acid amplification reaction involving a primer set in accordance with the invention and a target nucleic acid molecule is capillary electrophoresis (CE).

Thus, in accordance with a second aspect of the invention, there is provided an in vitro method for determining the human leukocyte antigen (HLA) genotype of a nucleic acid sample that has been obtained from a biological sample, comprising the steps of: providing at least one oligonucleotide primer set; contacting the nucleic acid sample with each of the primer sets and subjecting the nucleic acid sample and each primer set to a nucleic acid amplification reaction; separating any nucleic acid amplification products produced in each of the nucleic acid amplification reactions using a capillary electrophoresis (CE) separation technique; determining the size of the amplification products that have been separated using CE, and correlating the presence and/or absence of specific amplification products with the presence and/or absence of specific sequence polymorphisms associated with HLA alleles in the nucleic acid sample; and assigning an HLA genotype on the basis of the information derived from the presence and/or absence of the specific sequence polymorphisms associated with HLA alleles in the nucleic acid sample; wherein at least one of the at least one primer sets is a multi-specific primer set comprising at least one sequence-specific forward primer and at least one sequence-specific reverse primer and being adapted to amplify, in a nucleic acid amplification reaction, two or more specific target sequences that may be present in the nucleic acid sample, and wherein each of the specific target sequences comprises a sequence polymorphism that is known to be associated with an HLA allele and which may be present in the nucleic acid sample to be genotyped.

The benefits disclosed herein of using a capillary electrophoresis (CE) separation technique to resolve nucleic acid amplification products produced during the course of HLA genotyping also provide advantages over prior art HLA genotyping systems.

Thus, in a further aspect of the invention there is provided an in vitro method for determining the human leukocyte antigen (HLA) genotype of a nucleic acid sample that has been obtained from a biological sample, comprising the steps of: providing at least one oligonucleotide primer set; contacting the nucleic acid sample with the at least one primer set and subjecting the nucleic acid sample and at least one primer set to a nucleic acid amplification reaction; separating any nucleic acid amplification products produced in the nucleic acid amplification reaction using a capillary electrophoresis (CE) separation technique; determining the size of the amplification products that have been separated using CE, and correlating the presence and/or absence of specific amplification products with the presence and/or absence of specific sequence polymorphisms associated with HLA alleles in the nucleic acid sample; and assigning an HLA genotype on the basis of the information derived from the presence and/or absence of the specific sequence polymorphisms associated with HLA alleles in the nucleic acid sample; wherein each primer set comprises at least one sequence-specific forward primer and at least one sequence-specific reverse primer and is adapted to amplify, in a nucleic acid amplification reaction, two or more target sequences that may be present in the nucleic acid sample, and wherein at least one of the target sequences comprises a specific sequence polymorphism that is known to be associated with an HLA allele, and which may be present in the nucleic acid sample to be genotyped.

Preferably, at least one of the oligonucleotide primer sets is a multi-specific primer set.

In all aspects of the invention, the sample nucleic acid may be genomic DNA (gDNA) or cDNA (i.e. produced from mRNA expressed in a cell of a tissue type of interest) and may comprise one or more different nucleic acid molecules. Preferably, the sample nucleic acid molecule is gDNA.

Preferably, the biological sample of interest (i.e. that from which the nucleic acid sample is obtained) has been previously obtained from a subject, and is selected from the group consisting of epithelial tissue, blood, saliva, urine, semen, bone marrow, nasal fluid or tissue, and a hair follicle. The subject is preferably a human.

In the methods of the invention, the primer sets are preferably adapted to identify a specific allele of the HLA-A, HLA-B and HLA-DR genes. In order to genotype a highly polymorphic target locus, such as HLA, it is generally necessary to provide a plurality of different primer sets. However, it is preferable to minimise the number of different primer sets required to genotype the nucleic acid sample. Thus, preferably no more than 96 primer sets are provided. More preferably between 48 and 96 primer sets are required to identify the specific HLA allele. In some preferred embodiments approximately 48 primer sets may be provided.

In alternative embodiments of the invention, the at least one primer sets are adapted to identify a specific allele of the HLA-A, HLA-B, HLA-C, HLA-DR and HLA-DQ genes. The increased number of gene to be genotyped increases the number of potential polymorphisms. In this case, advantageously no more than 96 primer sets, and preferably less than 96 primer sets, such as between 48 and 96 primer sets are required to identify the specific HLA allele.

Thus, in view of the possible number of separate nucleic acid amplification reactions required to genotype a nucleic acid sample, it is preferable that the step of contacting the nucleic acid sample with the at least one primer set and subjecting the nucleic acid sample and at least one primer set to a nucleic acid amplification reaction is performed in an array, for example, in the form of a multi-well plate or lab-on-a-chip.

Preferably, the at least one sequence-specific forward primer and the at least one sequence-specific reverse primer of the multi-specific primer set constitute at least two specific primer pairs, each specific primer pair comprising a forward primer and a reverse primer and being adapted to amplify a specific target sequence that may be present in the nucleic acid sample. In preferred embodiments, the forward primer and/or the reverse primer of each specific primer pair is complementary to a specific sequence polymorphism that may be present in the nucleic acid sample to be genotyped, and wherein each of the specific primer pairs produces a specific amplification product only in the presence of the specific sequence polymorphism.

In all aspects of the invention, each of the specific target sequences preferably comprises at least one sequence polymorphism, the polymorphism being selected from the group comprising: single nucleotide polymorphisms (SNPs); insertions, substitutions and deletions of one or more nucleotides; and repetitive sequences (for example, microsatellites or repeats).

Preferably, the step of correlating the presence and/or absence of specific amplification products with the presence and/or absence of specific sequence polymorphisms in the nucleic acid sample is carried out in a computer using auto-interpretation software, and the software provides an output that reports the genotype information that has been derived on the basis of the presence and/or absence of the specific sequence polymorphisms.

In certain embodiments, at least one multi-specific primer set comprises two specific primer pairs, each specific primer pair being adapted to amplify a specific target sequence that may be present in the nucleic acid sample, and wherein each of the specific target sequences is in a different genetic locus of the nucleic acid sample. This arrangement of target sequences may be considered to be “inter-loci”. In other embodiments, the target sequences for specific primer pairs of the same primer set may be within the same gene. In this case, the target sequences are considered to be “intra-locus”. Intra-locus target sequences may overlap.

In preferred embodiments of all aspects of the invention, each of the primer sets further comprises a positive control primer pair, the control primer pair comprises a forward primer and a reverse primer, which primers are adapted to amplify a control sequence that is known to be present in the nucleic acid sample, and which control sequence is in a different gene or genes to those to be genotyped.

Suitably, primer sets for use in accordance with the invention may comprise one or more primers selected from the group comprising SEQ ID NOS. 1 to 18, as shown in FIG. 2.

In preferred embodiments and aspects of the invention, the nucleic acid amplification technique is the polymerase chain reaction (PCR), and more preferably, the nucleic acid amplification technique is PCR-SSP.

In any method of the invention, the CE nucleic acid separation technique may be selection from the group consisting of capillary zone electrophoresis (CZE), capillary gel electrophoresis (CGE), capillary isoelectric focusing (CIEF), isotachophoresis (ITP), electrokinetic chromatography (EKC), micellar electrokinetic capillary chromatography (MECC or MEKC), micro emulsion electrokinetic chromatography (MEEKC), non-aqueous capillary electrophoresis (NACE) and capillary electrochromatography.

A most preferred form of CE is capillary gel electrophoresis (CGE). Typically, a plurality of capillaries is used to resolve the products of each nucleic acid amplification reaction. Most preferably, the products from each nucleic acid amplification reaction are separated and analysed in a separate capillary.

In alternative aspects and embodiments of the invention, the products of the nucleic acid amplification reactions may be resolved using microfluidics lab-on-a-chip technology, such as either the Agilent 2100 or Agilent 5100 systems (Agilent Technologies, Inc. CA, USA). Preferably, the Agilent 5100 system is used when CE is not used.

Preferably, the step of determining the size of any nucleic acid amplification products, which typically includes the step of separating the nucleic acid amplification products, for example, using CE or lab-on-a-chip based technology (i.e. resolving of nucleic acid amplification products) is automated. In this way, the need for human intervention can be reduced from the time when which the nucleic acid amplification reaction has taken place until the time when the result of the amplification reaction is known. In some cases manual loading of the separating system (such as CE) is required, however, in more preferred embodiments human intervention may not be required even to load samples into the separating system. Accordingly, it is preferred that following a nucleic acid amplification reaction, resolution is achieved by loading a predetermined sample size of the reaction mixture onto the matrix of a CE system or lab-on-a-chip system (such as the Agilent 2100 or 5100 systems) by robotic means, without the need for human intervention.

The means of resolution is preferably also automated, such that human intervention is minimised or not required to conduct or control the means of nucleic separation and analysis. Thus, the reaction product(s) from each nucleic acid amplification reaction is/are detected and reported using automated means of nucleic acid detection, and computer software to convert each reading into a corresponding indication of the presence or absence of a particular nucleic acid amplification product of a particular size.

Preferably, the step of correlating the presence and/or absence of specific amplification products with the presence and/or absence of specific sequence polymorphisms associated with the HLA allele of interest in the nucleic acid sample is carried out in a computer using auto-interpretation software. The software preferably uses a means of data comparison, such as one or more look-up tables, to correlate the pattern of nucleic acid amplification products obtained (each of which is associated with one or more specific polymorphism) with the pattern that would be obtained for any known allele of interest, such an a specific HLA allele. Conveniently, one look-up table is used for each gene to be genotyped. The software preferably then provides an output which reports the genotype information that has been derived on the basis of the presence and/or absence of the specific sequence polymorphisms detected. The output is conveniently in the form of a specific named allele.

Accordingly, in another aspect, the invention provides an in vitro method for determining the human leukocyte antigen (HLA) genotype of a nucleic acid sample that has been obtained from a biological sample, comprising the steps of: (i) providing at least one oligonucleotide primer set; (ii) contacting the nucleic acid sample with each of the primer sets and subjecting the nucleic acid sample and each primer set to a nucleic acid amplification reaction; (iii) separating any nucleic acid amplification products produced in each of the nucleic acid amplification reactions using a capillary electrophoresis (CE) separation technique; (iv) determining the size of the amplification products that have been separated using CE, and correlating the presence and/or absence of specific amplification products with the presence and/or absence of specific sequence polymorphisms associated with HLA alleles in the nucleic acid sample; and (v) assigning an HLA genotype on the basis of the information derived from the presence and/or absence of the specific sequence polymorphisms associated with HLA alleles in the nucleic acid sample; wherein at least one of the at least one primer sets is a multi-specific primer set comprising at least one sequence-specific forward primer and at least one sequence-specific reverse primer and being adapted to amplify, in a nucleic acid amplification reaction, two or more specific target sequences that may be present in the nucleic acid sample, and wherein each of the specific target sequences comprises a sequence polymorphism that is known to be associated with an HLA allele and which may be present in the nucleic acid sample to be genotyped; and wherein steps (iv) and (v) are carried out using an auto-interpretation software program run on a computer, which auto-interpretation software program avoids the requirement for manual interpretation of data to assign an HLA genotype.

Once again, the advantages of using a CE separating technique and auto-interpretation software is equally applicable and beneficial in prior art systems for HLA genotyping (e.g. using PCR-SSP). Hence, the invention further provides an in vitro method for determining the human leukocyte antigen (HLA) genotype of a nucleic acid sample that has been obtained from a biological sample, comprising the steps of: (i) providing at least one oligonucleotide primer set; (ii) contacting the nucleic acid sample with each of the primer sets and subjecting the nucleic acid sample and each primer set to a nucleic acid amplification reaction; (iii) separating any nucleic acid amplification products produced in each of the nucleic acid amplification reactions using a capillary electrophoresis (CE) separation technique; (iv) determining the size of the amplification products that have been separated using CE, and correlating the presence and/or absence of specific amplification products with the presence and/or absence of specific sequence polymorphisms associated with HLA alleles in the nucleic acid sample; and (v) assigning an HLA genotype on the basis of the information derived from the presence and/or absence of the specific sequence polymorphisms associated with HLA alleles in the nucleic acid sample; wherein each primer set comprises at least one sequence-specific forward primer and at least one sequence-specific reverse primer and is adapted to amplify, in a nucleic acid amplification reaction, two or more target sequences that may be present in the nucleic acid sample, and wherein at least one of the target sequences comprises a specific sequence polymorphism that is known to be associated with an HLA allele, and which may be present in the nucleic acid sample to be genotyped; and wherein steps (iv) and (v) are carried out using an auto-interpretation software program run on a computer, which auto-interpretation software program avoids the requirement for manual interpretation of data to assign an HLA genotype.

Hence, in a further aspect of the invention there is provided a software program for assigning a human leukocyte antigen (HLA) genotype of a nucleic acid sample, which software program: (i) correlates the presence and/or absence of specific nucleic acid amplification products of expected size with the presence and/or absence of specific sequence polymorphisms in an HLA gene or genes using a means of data comparison; and (ii) assigns an HLA genotype on the basis of the information derived from the presence and/or absence of the specific sequence polymorphisms associated with HLA alleles in the nucleic acid sample.

Preferably, the means of data comparison in step (i) is one or more look-up table, and more preferably, a separate look-up table is used to assign an HLA genotype for each HLA gene. In a preferred embodiment, the software has means for assigning the genotype of the HLA-A, HLA-B and HLA-DR genes. In another preferred embodiment, the software program has means for assigning the genotype of the HLA-A, HLA-B, HLA-C, HLA-DR and HLA-DQ genes.

The invention also provides a kit for determining the human leukocyte antigen (HLA) genotype of a nucleic acid sample obtained from a biological sample comprising: at least one oligonucleotide primer set; and operating instructions in the form of a protocol for performing the genotyping method; wherein at least one of the at least one primer sets is a multi-specific primer set comprising at least one sequence-specific forward primer and at least one sequence-specific reverse primer and being adapted to amplify, in a nucleic acid amplification reaction, two or more specific target sequences that may be present in the nucleic acid sample, and wherein each of the specific target sequences comprises a sequence polymorphism that is known to be associated with an HLA allele and which may be present in the nucleic acid sample to be genotyped. Preferably, the operating instructions are in the form of a protocol for performing any one of the methods of the invention.

Preferably in kit embodiments of the invention the at least one oligonucleotide primer sets are arranged in an array.

The primer sets and primer pairs of the kit aspects and embodiments of the invention are advantageously adapted in the manner described with respect to the above methods. For example, at least one primer set may be a multi-specific primer set adapted to enable multi-specific PCR-SSP (as described herein), and optionally includes a positive control primer pair.

As above, each of the specific target sequences comprises at least one sequence polymorphism, the polymorphism being selected from the group comprising: single nucleotide polymorphisms (SNPs); insertions, substitutions and deletions of one or more nucleotides; and repetitive sequences (for example, microsatellites or repeats).

Preferably, the genotype to be determined is the MHC genotype, and more preferably, one or more of the HLA genes. Advantageously, the kit is adapted for assigning the genotype of the HLA-A, HLA-B and HLA-DR genes in a nucleic acid sample, and optionally also the HLA-C and HLA-DQ genes. Where the kit is adapted to genotype the HLA-A, HLA-B and HLA-DR genes, advantageously fewer than 96 primer sets are provided, and preferably no more than approximately 48 primer sets are provided. Similarly, where the kits of the invention are designed to enable the identification a specific allele of any or all of the HLA-A, HLA-B, HLA-C, HLA-DR or HLA-DQ genes, the kit preferably comprises no more than 96 primer sets, and more preferably between 63 and 78 primer sets.

In a preferred form, the kit embodiments of the invention further comprise at least one compartment for separately compartmentalising each of the at least one primer sets, and wherein each primer set is pre-aliquoted into a separate one of the compartments. Hence, conveniently there is a minimum of one such container for each primer set provided in the kit.

In preferred kits the at least one compartment is in the form of a 96-well plate, and each of the at least one primer sets is pre-aliquoted into a separate well of the 96-well plate. Alternatively, the at least one compartment is in the form of a 384-well plate, and each of the at least one primer sets is pre-aliquoted into a separate well of a 384-well plate. Preferably in kit embodiments of the invention, the primer sets are provided in dried form, preferably the primer sets are freeze dried or lyophilised.

Any kit according to the invention may further comprises a software program for automation of particular steps in the genotyping procedure. Such a software program may comprise any of the features of the software program aspects and embodiments of the invention.

### BRIEF DESCRIPTION OF THE FIGURES

FIG. 1 illustrates a DNA amplification reaction using two primer sets which target: (a) distal loci of the template DNA; and (b) target overlapping loci of the template DNA;

FIG. 2 provides sequence listings for primers used in accordance with preferred embodiments of the present invention;

FIG. 3 provides three individual electropherograms that were obtained following DNA amplification, separation and identification of DNA products for the purpose of typing a specific HLA-A allele as described in Example 1;

FIG. 4 provides three individual electropherograms that were obtained following DNA amplification, separation and identification of DNA products for the purpose of typing a specific HLA-A as described in Example 1;

FIG. 5 presents a single electropherogram that was obtained following multi-specific DNA amplification, separation and identification of DNA products for the purpose of typing a specific HLA-A allele in accordance with the present invention, as described in Example 2;

FIG. 6 presents a single electropherogram that was obtained following multi-specific DNA amplification, separation and identification of DNA products for the purpose of typing a specific HLA-A allele in accordance with the present invention, as described in Example 2;

FIG. 7 provides a set of four electropherograms that were obtained following DNA amplification, separation and identification of DNA products for the purpose of typing two different HLA alleles as described in Example 3;

FIG. 8 provides a single electropherogram that was obtained following multi-specific DNA amplification, separation and identification of DNA products for the purpose of typing two different HLA alleles in accordance with the present invention;

FIG. 9 is a photograph of the results of a SSP-PCR experiment for HLA genotyping, involving 96 PCR reactions and analysis by slab agarose gel electrophoresis as in the prior art; and

FIG. 10 is a flowchart showing the steps involved in the typical genotyping protocols of the prior art and according to the invention.

### DETAILED DESCRIPTION OF THE INVENTION

Prior to setting forth the invention, a number of definitions are provided that will assist in the understanding of the invention.

Oligonucleotides, Primers and Nucleic Acid Amplification

As used herein the term “primer” refers to short sequence-specific single stranded oligonucleotides that are designed to be complementary to a target sequence of the nucleic acid molecule, and serve as a primer for nucleic acid extension reactions, for use in DNA amplification methods, such as PCR. Preferably, the oligonucleotide primer is a single-stranded nucleic acid molecule of from 12 to 80 bases, more preferably from 15 to 60 bases, from 18 to 50 bases or from 20 to 40 bases in length. Most preferably, the oligonucleotide primer is a short single-stranded DNA molecule. The primer may comprise one or more modifications as per an oligonucleotide, below.

An “oligonucleotide” is a single or double stranded covalently-linked sequence of nucleotides in which the 3′ and 5′ ends on each nucleotide are typically joined by phosphodiester bonds. The oligonucleotide may be made up of deoxyribonucleotide bases (e.g. a DNA molecule) or ribonucleotide bases (e.g. an RNA molecule). Preferably, the oligonucleotide is a single-stranded DNA molecule, which may be manufactured synthetically in vitro or isolated from natural sources. The term oligonucleotide as used herein is not intended to distinguish over a “polynucleotide”, for example. Instead it is intended merely that the term is interpreted to the extent that it is defined herein. An oligonucleotide used in accordance with the invention may contain modified nucleotides (or nucleotide derivatives), for example, nucleotides that resemble the natural nucleotides, A, C, G, T and U, but which are chemically modified. Chemical modifications can be beneficial, for example, in increasing oligonucleotide stability and resistance to degradation by exo- and/or endonucleases. Thus, an oligonucleotide may contain a mixture of modified and natural nucleotides (e.g. one or more modified bases). In addition, or in the alternative, the backbone bonds of an oligonucleotide molecule may be chemically modified, e.g. to increase resistance to degradation by nucleases. A typical backbone modification is the change of one or more phosphodiester bonds to a phosphorothioate bond. In particular, the 5′ most nucleotides may be modified to include a selectable marker or label (e.g. a fluorescent or radioactive label), to improve detection and/or quantitation of the amount of PCR product produced therefrom. Suitable labels for conjugating to or incorporating into oligonucleotides are known to the person of skill in the art.

The term “primer pair” as used herein refers to a pair of primers, consisting of one forward and one reverse primer (relative to the nucleic acid sequence to be amplified), which are necessary and sufficient for the amplification of a target nucleic acid sequence, for example, using PCR. More specifically, the term “primer pair” refers to a specific combination of a forward and a reverse primer that are designed to amplify a target nucleotide sequence, such as an HLA allele. A primer pair that is designed to amplify a control nucleic acid sequence, for example, to demonstrate (by way of a product of known size) that a particular DNA amplification reaction has worked (i.e. a positive control), but which otherwise provides no information relating to genotype, may be termed a “control primer pair”. In contrast, a primer pair that, in the presence of its specific target sequence, produces an amplification product that provides information relating to genotype, for example, to identify a particular polymorphism, may be termed a “specific primer pair”. It should be understood, however, that the terms “control” and “specific” may not always be used in conjunction with the term “primer pair”, and in those cases, whether the primer pair has a control or a specific function will be evident from the context in which it is used. Thus, while it will be appreciated that a control primer pair must be capable of recognising specifically the control target sequence (see definition of “sequence-specific” below), the term “specific” as used herein in relation to a primer pair refers to oligonucleotide primers and their respective products that provide specific genotype information.

The term “sequence-specific” in the context of nucleotide sequence recognition/complementarity between a particular oligonucleotide primer and its target nucleic acid sequence, means that the oligonucleotide primer has sufficient complementary to its target sequence such that under the appropriate reaction conditions for nucleic acid extension or amplification, any nucleic acid extension or amplification initiated by the oligonucleotide primer at non-selected (i.e. non-target) sequences is insignificant in comparison to the nucleic acid extension or amplification from the target sequence. That is, any non-specific amplification products do not detract from the ability to clearly recognise the presence or absence of the specific band. Therefore, it is not necessary that the oligonucleotide primer has 100% complementarity with its target sequence: for example, one or two mismatches may be tolerated within the binding region. In addition, it may not be necessary that the 5′ end of the oligonucleotide primer is complementary to the nucleic acid template molecule. For example, the 5′ end can be overhanging and, for example, contain additional sequences or labels.

As used herein, the term “multi-specific”, for example in the context of a DNA extension or amplification reaction, such as a “multi-specific PCR” reaction, means that a plurality of specific PCR products may be produced irrespective of whether the PCR reaction concerned also produces a positive control PCR product using a control primer pair. The term “multi-specific PCR” is thus distinguished from the term “multiplex PCR”; in the sense that a multiplex PCR reaction may include two primer pairs, one of which produces a control product and one of which is intended to produce a specific product; whereas a multi-specific PCR reaction includes at least two specific primer pairs, which in the presence of an appropriate nucleic acid sample, are each capable of producing different specific products (which products are not positive control products); and in addition, may optionally include a control primer pair.

The term “primer set” as used herein refers to a plurality of primers, comprising all of the primers sufficient to determine at least one polymorphism within a target genetic locus of a sample nucleic acid molecule. More specifically, a primer set includes at least one forward primer and at least one reverse primer, provided that the primer set contains at least three different primers; and that the primer set is capable, in the presence of the appropriate sample nucleic acid molecule, of producing at least two amplification products (e.g. PCR products). By way of further explanation, it will be appreciated that a primer set may produce two amplification products where a single forward primer is capable of amplifying a target nucleic acid sequence in conjunction with either of two different reverse primers; or where a single reverse primer is capable of amplifying a target nucleic acid sequence in conjunction with either of two different forward primers. In each of the above examples, there are two “primer pairs”, but only three different oligonucleotide primers. Preferably, however, a primer set that is capable (in the presence of the appropriate sample nucleic acid molecule), of producing at least two amplification products includes at least two forward primers and at least two reverse primers; e.g. a primer pair consisting of forward primer “a-for” and reverse primer “a-rev”, capable of producing a first amplification product (A), and a second different primer pair consisting of forward primer “b-for” and reverse primer “b-rev”, capable of producing a second amplification product (B). The target sites for the respective primer pairs (a and b, for example) may be within the same locus of the sample nucleic acid molecule, in which case the combination is said to be “intra-loci”; or the target sites may be in different loci, in which case the combination is said to be “inter-loci”. In an intra-loci reaction, the target sites or the respective nucleic acid amplification products may even overlap.

Thus, each primer set for use in accordance with the invention is capable of directing the amplification of at least two nucleic acid sequences in the presence of an appropriate sample nucleic acid sequence. In other words, where the sample nucleic acid molecule(s) comprise(s) target sequences for each of the primers in a particular primer set, then more than one (e.g. two) nucleic acid amplification products will result. Thus, each primer set as defined herein is adapted to enable multiplex PCR (provided the respective target sequences are present in the sample nucleic acid).

A “multi-specific primer set” is a primer set that is adapted to enable multi-specific DNA extension or amplification reactions, preferably multi-specific PCR. Hence, a multi-specific primer set comprises at least two specific primer pairs, which in the presence of a suitable nucleic acid sample, can produce at least two specific amplification products. However, it will be appreciated that when the nucleic acid sample does not contain each of the specific target sequences (e.g. specific sequence polymorphisms) that are recognised by one or more of the primers of a primer set, one or more of the possible specific nucleic acid products can not be produced in the multi-specific PCR reaction.

Each multi-specific primer set is adapted to enable the amplification of two or more specific nucleic acid sequences within a nucleic acid sample; e.g. 2, 3 or 4 specific nucleic acid sequences. Preferably, each multi-specific primer set is adapted to amplify two specific nucleic acid sequences within a nucleic acid sample. In addition, each multi-specific primer set preferably includes a control primer pair for confirming that the nucleic acid amplification reaction has worked, for example, in the event that no specific target nucleic acid sequences (corresponding to genetic polymorphisms) are detected. Preferably, therefore, a multi-specific primer set for use in accordance with the invention comprises two specific primer pairs and a control primer pair.

As part of a kit or method for determining the genotype of a nucleic acid sample in accordance with the invention it is advantageous that a plurality of primer sets are provided; i.e. two or more primer sets. Preferably, the plurality of primer sets includes at least one multi-specific primer set. It is advantageous that the plurality of primer sets includes at least 10%, at least 20% or at least 30% multi-specific primer sets. More preferably, the plurality of primer sets includes a majority of multi-specific primer sets; for example at least 50%, at least 60% or at least 70% multi-specific primer sets. Most preferably, the plurality of primer sets comprises at least 80%, at least 90% or at least 95% multi-specific primer sets.

Where a plurality of multi-specific primer sets are provided, the plurality of multi-specific primer sets may include multi-specific primer sets that are adapted to amplify two specific nucleic acid sequences, as well as multi-specific primer sets that are adapted to amplify three specific nucleic acid sequences. The plurality of multi-specific primer sets may also include multi-specific primer sets that are adapted to amplify four or more specific nucleic acid sequences. It is advantageous that the majority of the plurality of multi-specific primer sets are adapted to amplify two specific nucleic acid sequences. Thus, a minority of multi-specific primer sets of the plurality may be adapted to amplify three specific nucleic acid sequences, and still other multi-specific primer sets may be adapted to amplify four specific nucleic acid sequences.

It will be appreciated that to enable the different specific amplification products to be distinguished, it is preferable that the products have different sizes, i.e. different numbers of nucleotide base pairs. It will be appreciated that the requirement for a particular difference in product size will be dependent on the resolution limit of the means of detection. Thus, if the products are to be resolved by gel electrophoresis, for example, the resolution may be dependent on both the absolute size of the products (e.g. 200 bps or 2 kbps), and the actual difference in size between the respective products (e.g. 20 or 40 bps difference). Preferably, the difference in product size between each product generated by (or capable of being generated by) a primer set is at least 10 bps, more preferably at least 15 bps and most preferably, at least 20 bps.

It is preferable that where a primer set or multi-specific primer set includes a control primer pair, the positive control product is the largest of any of the nucleic acid amplification products that is capable of being produced by that primer set. In this way the nucleic acid amplification reaction can be more conveniently adapted to promote efficient production of specific nucleic acid amplification products. For the reasons given above, the control product should be at least 10 bps, preferably at least 15 bps and more preferably at least 20 bps larger that the largest possible specific nucleic acid product producible from the respective primer set.

In the alternative, one or both of the oligonucleotide primers of each primer pair may be labelled (e.g. using a fluorescent marker), such that each of the nucleic acid products is distinguishable from the other. In this way, different products may be detected and/or resolved and/or quantified without the need for a particular difference in size between the products. Thus, the difference in product size between each specific product produced by a primer set may be less than 20 bps, less than 15 bps or even less than 10 bps.

Genotyping and Polymorphism

The term “genotype” as used herein refers to the version of the gene, or allele, carried by a test subject (individual or patient), as well as any further genetic information associated therewith. The genetic information of most interest in the context of the invention is the genotype of the MHC region. More preferably, the genotype of interest is the HLA region in human DNA.

The term “polymorphism” refers to the genetic variation that that is found within individuals of the same species. For example, different hair colour in humans is an example of polymorphism in the genes associated with determining hair colour. Hair colour is an example of a polymorphism that produces a phenotype; i.e. a visible trait. However, many types of polymorphism result in no discernable difference between individuals of a species, except at the level of nucleic acid (genetic DNA) sequence. Typical examples of polymorphisms that do not produce a phenotype include single nucleotide polymorphisms (SNPs) and restriction fragment length polymorphisms (RFLPs).

Polymorphisms may occur in coding or non-coding regions of the HLA genes. For example, polymorphisms can occur in upstream untranslated 5′ regions, introns, exons, downstream 3′ regions, enhancer regions, promoter regions and/or in a region known to be involved in epigenetic or other DNA modifying activity. SNPs, insertions, substitutions and deletions of one or more nucleotides and repetitive sequences (microsatellites or repeats) are all examples of polymorphisms that can contribute to allelic variations in a population. Depending on where in the genome these polymorphisms occur, they may or may not affect the biological activity of the protein encoded by the gene. Nevertheless, identification of these polymorphisms is critical in determination of genotype, particularly with respect to the HLA genes.

Accordingly, nucleic acid target sequences for oligonucleotide primers used in accordance with the invention are preferably sequences (within a nucleic acid sample) in which polymorphisms have been identified or are known to occur; such that a sequence-specific oligonucleotide primer is capable of directing nucleic acid extension and/or amplification (in combination with a second primer) only in the presence of nucleic acid sequences that are specific for a particular genetic polymorphism. The polymorphism relates to the MHC genomic locus, and preferably to a specific HLA allele.

By contrast, a control sequence for amplification by a pair of control primers is a sequence of known length, which sequence is not associated with a polymorphism of interest and which is preferably within a gene (or other genetic sequence) entirely unrelated to the genes of interest.

A nucleic acid sample for use in accordance with the invention is derived, obtained or extracted from cells within a selected tissue sample. Hence, the nucleic acid sample is typically genetic DNA and, consequently, the sample includes more than one nucleic acid molecule; for example, corresponding to different chromosomes within the sample.

It will be appreciated that the selected tissue sample can be any tissue of interest: for example, epithelial tissue (skin), muscle, bone (marrow), blood; or it can be any organ, such as liver, kidney, lung or heart tissue.

HLA SSP Genotyping

The methods and systems of the invention are adapted to genotype the HLA region using the sequence-specific primer PCR (PCR-SSP) method. As already discussed, an advantage of SSP is that, because the oligonucleotide primers are designed to complement specific polymorphisms, only nucleic acid sequences in the nucleic acid sample that contain that specific allele (polymorph) should be amplified. However, as discussed below, the extent of polymorphism of the target gene(s) can make genotyping of certain genes highly burdensome.

HLA genes are highly polymorphic. The SSP method requires a panel of PCR reactions, each containing primers to detect specific polymorphisms. In view of the extent of polymorphism of the HLA-B locus, for example, in prior art methods 48 separate PCR reactions are required for typing the B locus alone. Each of these 48 PCR reactions contains a specific primer pair to detect a specific sequence polymorphism. If the specific polymorphism is present, the PCR reaction will generate a specific PCR product, which will typically be visualised as a band on the gel. On the other hand, if the specific polymorphism is absent, the specific PCR product will not be generated. For each of the 48 PCR reactions, the reaction will be scored positive or negative according to whether the expected specific PCR product is produced. This results in a combination of 48 positive or negative scores and, based on the combination of the positive reactions the genotype of the HLA-B locus can be assigned; typically by reference to a worksheet.

In addition to the specific primer pairs for amplifying specific polymorphisms, each reaction generally also contains a positive control primer pair to demonstrate that each PCR reaction was successful. The positive control provides valuable information on the quality of the test, especially in circumstances where the specific polymorphism associated with the specific primer pair in a reaction is absent. Typically, the control primers amplify a conserved region of a house-keeping gene in the nucleic acid sample. Thus, where the tissue sample is from a human, the control primers would target a nucleic acid sequence (or gene) that is present in all human DNA samples.

It should be noted that the prior art process outlined above only provides genotype information relating to the HLA-B allele.

Prior art genotyping methods for the HLA-A and HLA-DR genes operate in a similar manner to that for HLA-B discussed above. However, in each case another 24 separate PCR-SSP reactions are necessary to identify a specific HLA-A or HLA-DR allele (see for example Table 1, which provides a prior art look up table for HLA-A). Each of these reactions should preferably also include a positive control PCR reaction.

Accordingly, it is necessary to run at least 96 separate PCR-SSP reactions (each including an internal control), in order to identify the combination of HLA-A, -B and -DR alleles in a test sample.

| | A* alleles | Primer Mix Number | | | | | | | | | | | | |
|---|---|---|---|---|---|---|---|---|---|---|---|---|---|---|
| Serology | amplified with | 1 | 2 | 3 | 4 | 5 | 6 | 7 | 8 | 9 | 10 | 11 | 12 | 13 |
| Type | A primer mix | 95 | 165 | 170 215 | 180 | 195 | 215 220 | 215 175 | 90 | 80 70 | 200 | 185 | 205 | 95 |
| A1 | A*0101-03/06-08 | | | | | | | | | | | | | |
| A1 | A*0109 | | | | | | | | | | | | | |
| A2 | A*020101-0109/03/04/ | | 2 | | 4 | | | | | | | | | |
| | 06/07/09-13/16/18/ | | | | | | | | | | | | | |
| | 20-22/24-31/33-38/ | | | | | | | | | | | | | |
| | 40-42/45/46/48/49/ | | | | | | | | | | | | | |
| | 51/52/54-56/59/60 | | | | | | | | | | | | | |
| A2 | A*0202/05/08/14/15N/ | | 2 | | | | | | | | | | | |
| | 17/19/23/32N/39/43N/ | | | | | | | | | | | | | |
| | 44/47/50/53N/57/58 | | | | | | | | | | | | | |
| A3 | A*030101-0103/04-10 | | | 3 | | | | | | | | | | |
| A3 | A*0302 | | | 3 | | | 6 | | | | | | | |
| A11 | A*1101-10/12-14 | | | | | | 6 | | | | | | | |
| A11 | A*1111 | | | | | | 6 | | | | | | | |
| A23 | A*2301-06 | | | | | 5 | 6 | | | | | | | |
| A23 | A*2309 | | | | | 5 | | | | | | | | |
| A24 | A*24020101-07/10/ | | | | | | 6 | 7 | | | | | | |
| | 13-15/17-23/25-30/ | | | | | | | | | | | | | |
| | 32-35/37/38 | | | | | | | | | | | | | |
| A24 | A*2408/31 | | | | | | | 7 | | | | | | |
| A24 | A*2424 | | | | | 5 | | | 8 | | | | | |
| A25 | A*2501/03/04 | | | | | | | | 8 | | | | | |
| A25 | A*2502 | | | | | | | | 8 | | | | | |
| A26 | A*2601-08/10/12-18 | | | | | | | | | 9 | | | | |
| A26 | A*2609 | | | | | | | | | 9 | | | | |
| A29 | A*2901-06 | | | | | | | | | | 10 | | | |
| A29 | A*2907 | | | | | 5 | | | | | 10 | | | |
| A30 | A*3001-04/06/09-12 | | | | | | | | | | | 11 | | |
| 30 | A*3007 | | | | | | 6 | | | | | 11 | | |
| A30 | A*3008 | | | | | | | | | | | 11 | | |
| A31 | A*310102-06/08-09 | | | | | | | | | | | | 12 | |
| A31 | A*3107 | | | | | | | | | | | | 12 | 13 |
| A32 | A*3201-07 | | | | | | | | | | | | | 13 |
| A32 | A*3204 | | | 3 | | | | | | | | | | 13 |
| A33 | A*3301-06 | | | | | | | | | | | | | |
| A34(10) | A*3401-05 | | | | | | | | | | | | | |
| A36 | A*3601/03/04 | | | | | | | | | | | | | |
| A36 | A*3602 | | | 3 | | | | | | | | | | |
| A43 | A*4301 | | | | | | | | | | | | | |
| A66(10) | A*6601/04 | | | | | | | | | | | | | |
| A66(10) | A*6602 | | | | | | | | | | | | | |
| A66(10) | A*6603 | | | | | | | | | | | | | |
| A68(28) | A*680101-02/06-10/ | | | | | | | | | | | | | |
| | 12-14/16/17/19/ | | | | | | | | | | | | | |
| | 21-23 | | | | | | | | | | | | | |
| A68(28) | A*6803/04/11 | | | | | | | | | | | | | |
| A68(28) | A*6805/15/20 | | | | | | | | | 9 | | | | |
| A69(28) | A*6901 | | | | 4 | | | | | | | | | |
| A74(19) | A*7401-09 | | | | | | | | | | | | | |
| A80 | A*8001 | | | | | | | | | | | | | |
| C. C | DNA & Carry-over | 1 | | | | | | | | | | | | |
| | Contamination | | | | | | | | | | | | | |
| | Control | | | | | | | | | | | | | |

| | A* alleles | Primer Mix Number | | | | | | | | | | |
|---|---|---|---|---|---|---|---|---|---|---|---|---|
| Serology | amplified with | 14 | 15 | 16 | 17 | 18 | 19 | 20 | 21 | 22 | 23 | 24 |
| Type | A primer mix | 205 | 170 | 170 | 200 | 175 | 175 | 180 210 | 230 | 155 | 170 | 175 |
| A1 | A*0101-03/06-08 | | | 16 | | | | | | | | 24 |
| A1 | A*0109 | | | | | | | | | | | 24 |
| A2 | A*020101-0109/03/04/ | | | | | | | | | | | |
| | 06/07/09-13/16/18/ | | | | | | | | | | | |
| | 20-22/24-31/33-38/ | | | | | | | | | | | |
| | 40-42/45/46/48/49/ | | | | | | | | | | | |
| | 51/52/54-56/59/60 | | | | | | | | | | | |
| A2 | A*0202/05/08/14/15N/ | | | | | | | | | | | |
| | 17/19/23/32N/39/43N/ | | | | | | | | | | | |
| | 44/47/50/53N/57/58 | | | | | | | | | | | |
| A3 | A*030101-0103/04-10 | | | | | | | | | | | |
| A3 | A*0302 | | | | | | | | | | | |
| A11 | A*1101-10/12-14 | | | | | | | | 21 | | | |
| A11 | A*1111 | | | | 17 | | | | 21 | | | |
| A23 | A*2301-06 | | | | | | | | | | | |
| A23 | A*2309 | | | | | | | | | | | |
| A24 | A*24020101-07/10/ | | | | | | | | | | | |
| | 13-15/17-23/25-30/ | | | | | | | | | | | |
| | 32-35/37/38 | | | | | | | | | | | |
| A24 | A*2408/31 | | | | | | | | | | | |
| A24 | A*2424 | | | | | | | | | | | |
| A25 | A*2501/03/04 | | | | | 18 | | | | | | |
| A25 | A*2502 | | | | | 18 | | | 21 | | | |
| A26 | A*2601-08/10/12-18 | | | | | 18 | | | | | | |
| A26 | A*2609 | | 15 | | | | | | | | | |
| A29 | A*2901-06 | | | | | | | | | | | |
| A29 | A*2907 | | | | | | | | | | | |
| A30 | A*3001-04/06/09-12 | | | | | | | | | | | |
| 30 | A*3007 | | | | | | | | | | | |
| A30 | A*3008 | | | | | | | | 21 | | | |
| A31 | A*310102-06/08-09 | | | | | | | | | | | |
| A31 | A*3107 | | | | | | | | | | | |
| A32 | A*3201-07 | | | | | | | | | | | |
| A32 | A*3204 | | | | | | | | | | | |
| A33 | A*3301-06 | 14 | | | | | | | | | | |
| A34(10) | A*3401-05 | | 15 | | | | | | 21 | | | |
| A36 | A*3601/03/04 | | | 16 | | | | | | | | |
| A36 | A*3602 | | | 16 | | | | | | | | |
| A43 | A*4301 | | | | 17 | 18 | | | | | | |
| A66(10) | A*6601/04 | | | | | 18 | | | 21 | | | |
| A66(10) | A*6602 | | | | | | 19 | | 21 | | | |
| A66(10) | A*6603 | | | | | | 19 | | | | | |
| A68(28) | A*680101-02/06-10/ | | | | | | | 20 | 21 | | | |
| | 12-14/16/17/19/ | | | | | | | | | | | |
| | 21-23 | | | | | | | | | | | |
| A68(28) | A*6803/04/11 | | | | | | | 20 | | | | |
| A68(28) | A*6805/15/20 | | | | | | | 20 | | | | |
| A69(28) | A*6901 | | | | | | | | 21 | | | |
| A74(19) | A*7401-09 | | | | | | | | | 22 | | |
| A80 | A*8001 | | | | | | | | | | 23 | |
| C. C | DNA & Carry-over | | | | | | | | | | | |
| | Contamination | | | | | | | | | | | |
| | Control | | | | | | | | | | | |

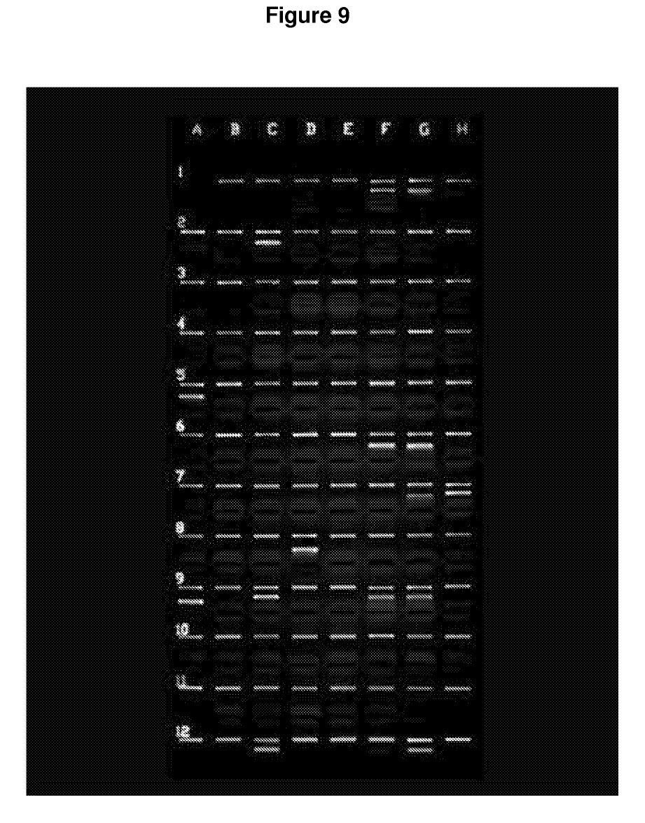

A typical result from a prior art HLA-SSP typing test is shown in FIG. 9. As depicted, the products from each of the 96 PCR-SSP reactions are separated on a 96-well slab gel. The PCR product from the internal control primers is larger than those of the specific primer pairs. In the example depicted, the first reaction (top left-hand corner) is a contamination control, and reactions 6, 7, 11, 33, 46, 47, 55, 56, 60, 65, 67, 70, 71, 91 and 95 are scored positive.

Multiplex and Multi-Specific PCR-SSP

Multiplex PCR is a variant of PCR that enables simultaneous amplification of more than one target sequence in one reaction by using more than one pair of primers. Prior art genotyping systems may already include multiplex PCR reactions to the extent that each PCR reaction includes a specific primer pair for identifying a specific polymorphism and a positive internal control primer pair.

In contrast to the prior art, the present invention relates to “multi-specific” PCR, in which at least one PCR reaction of the HLA SSP typing test is capable of generating more than one specific PCR product when the target polymorphisms are present.

Thus, the invention relates to specially adapted primer sets (“multi-specific PCR sets”) for PCR-SSP, which are capable of identifying more than one specific polymorphism at the same time. That is, by adding a particular multi-specific primer set to a nucleic acid sample, potentially more than one (for example, 2, 3 or 4; preferably 2) polymorphisms can be identified. In this way, the extremely large number of separate PCR reactions that were previously necessary for an HLA-A, -B and -DR genotyping test are reduced.

In accordance with one embodiment of the invention using multi-specific PCR design, no more than 96 PCR reactions are required to achieve HLA-A, -B and -DR typing. Preferably, these 96 reactions may be capable of identifying more than 96 different HLA polymorphisms. In preferred embodiments at least one primer set is designed to generate two specific products of different size in a single PCR-SSP reaction, i.e. at least one primer set is a multi-specific primer set. In particularly advantageous embodiments at least 10%, at least 20%, or at least 30% of the PCR-SSP reactions conducted generate more than one specific product, each of which can be used to identify a specific polymorphism in HLA. In particularly preferred embodiments a majority of primer sets are multi-specific primer sets. In this way, the number of primer sets required to genotype HLA-A, -B and -DR can be reduced from the 96 primer sets of the prior art. Accordingly, it is preferable that less than 96 PCR reactions are required to achieve HLA-A, -B and -DR typing; more preferably between 48 and 96 PCR reactions are required; and in some especially preferred embodiments only approximately 48 PCR reactions are required.

In designing suitable multi-specific primer sets for use in multi-specific PCR it is important to consider the problems that could be associated with mixtures of several PCR primers in a PCR reaction. For example, the skilled person in the art will appreciate that the more primers there are in a single PCR reaction, potentially the more difficult it is to optimise the reaction; to avoid primer competition and non-specific amplification. In addition, it is necessary to consider the sizes of the amplification products that are expected to result from each primer pair. As already discussed, the primer sets are designed such that the products sizes are preferably at least 20 bps apart.

Multi-specific PCR as used in accordance with the invention can involve either or both of intra-locus and inter-loci combinations.

By way of example, the prior art genotyping of HLA-B requires 48 separate PCR reactions, as already discussed. By using intra-locus multi-specific PCR design (i.e. where all specific primers in each primer set are designed to target polymorphisms within the HLA-B gene), the number of PCR-SSP reactions required to genotype HLA-B is reduced to less than 48 reactions, and preferably to about 24 reactions; for example, when the specific primers in each primer set target on average two specific polymorphisms at a time. As previously discussed, however, some multi-specific primer sets may be adapted to generate two specific amplification products, while others may be adapted to generate more than two specific amplification products.

In the case of multi-specific PCR design with inter-loci combination, the specific primers are capable of targeting polymorphisms within different loci, such as HLA-A and HLA-B. For example, the total number of PCR reactions to type the full HLA-A, -B and -DR genes can be reduced from a total of 96 reaction in the prior art to 48 or less. In inter-loci PCR-SSP, multi-specific primer sets may be designed to target polymorphisms in the A and B loci, the B and DR, or the A and DR loci.

In addition to genotyping the HLA-A, -B, and -DR genes, full HLA genotyping may also involve identification of HLA-C and HLA-DQ alleles in the nucleic acid sample. Conventionally, 22 separate PCR-SSP reactions are required to genotype the HLA-C gene and 8 separate PCR-SSP reactions are required to identify the particular HLA-DQ allele in a genetic sample (i.e. 30 reactions in total). Therefore, to identify all of the specific HLA-A, -B, -C, -DR and -DQ alleles in a genetic sample, at least 126 separate PCR-SSP reactions (each including a positive control) are required.

Clearly, such a large number of reactions are labour intensive in terms of experimental set-up, and are also onerous and complicated in terms of data analysis. These problems are exacerbated when it is considered that these 126 reactions cannot be carried out in a conventional single 96-well plate format. The use of two separate 96-well plates in order to analyse a single genetic sample further increases the costs of operating the genotyping system, doubles the time taken to analyse a single sample, and greatly increases the chance of making a mistake in the data analysis.

The invention solves many of the problems associated with additionally genotyping the HLA-C and HLA-DQ genes, because using multi-specific PCR-SSP, the total number of reactions required to genotype the HLA-A, -B, -C, -DR and -DQ alleles from a nucleic acid sample is reduced to 96 separate reactions or less. For example, multi-specific PCR-SSP for genotyping the HLA-A, -B, and -DR alleles requires less than 96, such as only 48 primer sets (and hence only 48 separate PCR reactions). Therefore, even with the additional 30 conventional PCR-SSP reactions necessary for genotyping both HLA-C and HLA-DQ, the combined reactions may be analysed in a single 96-well plate format by using the methods and kits of the invention. Moreover, by creating primer sets for multi-specific PCR of HLA-C and HLA-DQ, the total number of reactions required to genotype HLA-C and HLA-DQ can be reduced to below 30 reactions, for example, 25 or less, 20 or less or 15 reactions (or less). In this way, the HLA-A, -B, -C, -DR and -DQ genes can be fully genotyped using 96 primer sets or less and, therefore, 96 or less separate PCR-SSP reactions.

Thus, the benefits achieved by the invention over prior art systems for genotyping HLA, may allow analysts to routinely genotype for all of the HLA-A, -B, -C, -DR and -DQ genes in a genetic sample, rather than the currently more limited approach of genotyping the HLA-A, -B, and -DR genes only.

It is notable that, when HLA genotyping in accordance with the invention is carried out in 384-well format, for example, in combination with the Agilent 5100 system (see below), then many different nucleic acid samples can be fully genotyped on a single 384-well plate by using the methods of the invention.

In a method, system or kit according to the invention for genotyping the HLA genes (for example, HLA-A, -B and -DR genes, and optionally the HLA-C and -DQ genes), in order to optimally reduce the total number of reactions required, primer sets may be provided for both intra-locus and inter-loci multi-specific PCR.

Thus, in accordance with the invention, genotyping throughput and especially HLA-genotyping throughput can be greatly increased using multi-specific PCR design.

Analysis of PCR-SSP Results

The invention preferable relates to systems for analysing the results of multi-specific PCR reactions that can be readily and reliably automated, such as capillary electrophoresis (CE) and the Agilent 2100 or 5100 systems.

The term “capillary electrophoresis” (CE) is used herein to describe a family of related separation techniques that use narrow-bore capillaries to separate biological molecules such as DNA fragments in a support medium under a high strength electric field. CE has been described in detail in Kemp, G., 1998. Capillary electrophoresis: a versatile family of analytical techniques. Biotechnol. Appl. Biochem., 27, pp 9-17. Separation of molecules is typically achieved according to differences in size, charge, and hydrophobicity of the molecules. A number of forms of CE exist, classified depending upon the type of capillary and electrolytes used, including, for example: capillary zone electrophoresis (CZE); capillary gel electrophoresis (CGE); capillary isoelectric focusing (CIEF); isotachophoresis (ITP); electrokinetic chromatography (EKC); micellar electrokinetic capillary chromatography (MECC or MEKC); micro emulsion electrokinetic chromatography (MEEKC); non-aqueous capillary electrophoresis (NACE); and capillary electrochromatography. It will be appreciated by one skilled in the art that any of these techniques may be more or less suitable for the separation of DNA products following a nucleic acid amplification reaction.

CGE is a particularly suitable form of CE for separation of nucleic acid molecules of different sizes. CGE is a technique that is known to the skilled person in the art. However, in brief: charged nucleic acid molecules, such as DNA, are separated by filling a capillary with a gel matrix and applying a potential difference along the capillary; longer nucleic acid strands are retarded by the matrix to a greater extent than shorter strands, such that the longer the strand, the slower it migrates along the capillary; nucleic acid products are detected as they travel along the capillary past a detection means.

CE techniques are advantageously used in accordance with the invention, because CE is typically straightforward to perform, can be readily automated, and offers short separation times. Thus, CE offers significant savings in time and skilled operator input compared to traditional slab gel formats such as agarose gel electrophoresis. In addition, as the capillary volume is generally not more than a few nanolitres, the running costs are typically lower than those of slab gel techniques; the use of capillaries allows the use of simpler nucleic acid detection means; and importantly, CE can provide higher resolution than slab gel electrophoresis.

Furthermore, in contrast to slab gel electrophoresis, the gels used in CE may not be present as a single solid piece and, instead, may comprise several separate segments of matrix. A single block of matrix (e.g. gel) is not necessary in CE (or CGE) as the capillary supports the matrix. In fact, it is possible for the matrix used in CE to be a liquid polymer. This provides the advantage that the matrix can be readily replaced between separate runs, and hence, cross-contamination can be reduced.

In summary, PCR-SSP genotyping using CE and automation (for example, using robotic arms and other manipulation equipment), is far more reproducible, reliable and accurate than the use of slab gel alternatives. By using automation, the present invention eliminates the possibility of human error in loading PCR samples for analysis and in reading/analysing those samples.

The Agilent 2100 and Agilent 5100 systems are, like CE, preferred alternatives to slab electrophoresis. The Agilent 2100 and Agilent 5100 systems are fully automated and hence, reproducible, and provide digital output data. These systems automate the entire electrophoresis procedure including sample handling and data analysis. Using the Agilent 5100 system, up to 3,840 samples (i.e. 10×384 well plates) can be analysed in each run. The Agilent 5100 system can operate in 96- or 384-well plate format. Samples are loaded onto a microfluidic chip for separation and detection using an automated, robotic handling mechanism. An incorporated software system analyses the results of each reaction sample and generates digital output data.

Computer-Aided Data Analysis (Software)

The steps involved in prior art HLA SSP genotyping tests typically include: (i) PCR-SSP (to test for one polymorphism at a time); (ii) manual agarose (slab) gel electrophoresis; (iii) manual obtaining of raw data from the gel; (iv) manual interpretation and input of raw data into a data processing means or manual comparison with a typing table; and (v) determining the typing result.

Conventionally, therefore, after the PCR step, the PCR reactions are resolved by agarose gel electrophoresis. The raw data obtained is typically one or more gel photos, for example, as shown in FIG. 9. The data has to be interpreted manually to decide which reactions are scored as positive and which are negative. After the combination of positive reactions is determined, the HLA genotype can be worked out using a worksheet (as shown in Table 1) or using computer software. However, even when computer software is used to provide the genotype output, the conventional software requires a manual input of raw data corresponding to the combination of positive reactions, before the genotype results can be generated. In other words, the software merely replaces the alternative process of comparing the raw data with the worksheet. There are a number of disadvantages that are associated with such prior art procedures, which include: (1) manual interpretation of the gel (raw data) involves subjective determination of positive reactions; (2) manual interpretation of raw data is time consuming and can be a rate-limiting step in high-throughput genotyping operations; (3) manual input of the combination of positive reactions (raw data) into a data processing means is time consuming; and (4) manual input of raw data into a data processing means carries a significant risk of human errors; which could involve more time-consuming checking of the data, or worse, lead to a wrong typing result.

In accordance with preferred embodiments and aspects of the present invention, computer software is provided that eliminates the problems associated with prior art systems for data analysis to obtain a genotyping result.

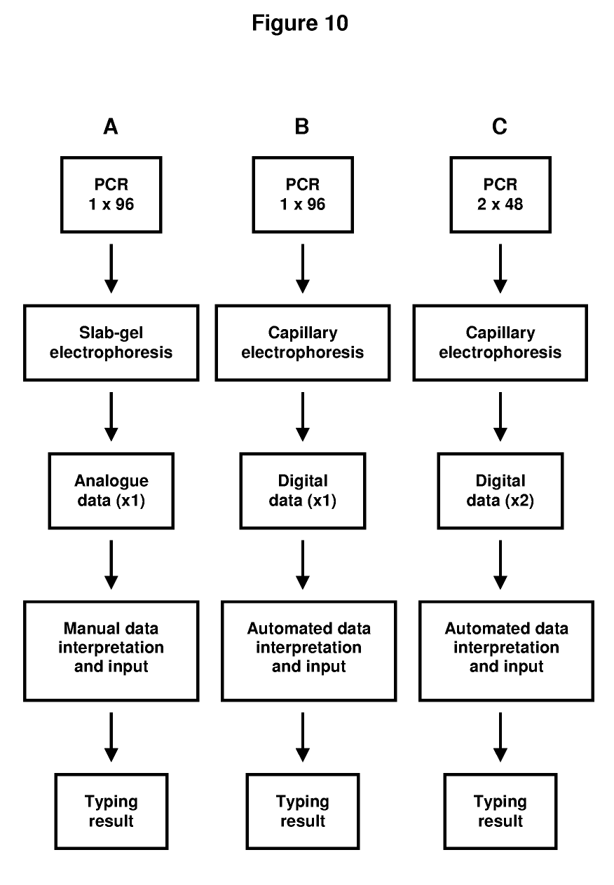

FIG. 10 is a flow diagram illustration to demonstrate the differences between the automated software genotyping systems of the invention and the prior art.

Column A of the flowchart illustrates the procedure used in existing PCR-SSP genotyping technology; column B represents a genotyping protocol employing CE technology to analyse PCR-SSP samples; and column C represents a genotyping protocol employing multi-specific PCR and CE technology to analyse PCR-SSP samples. Columns B and C represent protocols that may be used in accordance with the invention.

In the first stage (top row), it can be seen that in the protocol of column C, due to the benefits of multi-specific PCR, two sets of genetic samples for HLA-A, HLA-B and HLA-DR genotyping can be tested at once, with the same total number of PCR-SSP reactions (i.e. 96). In column C it is not necessary that all of the PCR reactions are adapted for multi-specific PCR, nor that each multi-specific PCR reaction is adapted to recognise exactly two specific target sequences. The important feature is that the total number of PCR reactions for the genotyping is reduced.

In the second row (Electrophoresis) the prior art process of column A typically uses manually loaded agarose slab gel electorphoreses to resolve amplified nucleic acid products from each PCR-SSP reaction. Loading of slab gels is not suited to automation and is error prone. In contrast, the two processes of the invention (columns B and C) use CE to separate the products of the PCR-SSP reactions. Accordingly, the processes of the invention eliminate the possibility of human error in loading samples and also provide a faster, more reliable and more accurate system for resolving the products of PCR-SSP.

The third row (data) indicates that the prior art protocol generates an analogue gel image, which is stored as a picture or digital file. The resolution of the gel is typically quite low and can be messy; leading to a possibility of selecting false-positive and/or false-negative results. In contrast, the protocols used in accordance with the invention generate either one or two sets (respectively) of digital data, which are easily quantified and any potential false-positive or false-negative readings are resolved by the auto-interpretation software employed to read the CE results.

The fourth row (data interpretation) indicates that in the prior art system, once a photograph of an agarose gel displaying the results of PCR-SSP has been obtained, a user must then typically manually input the data into accessory software. In the systems of the invention, however, any manual steps in the determination and interpretation of data are minimised or eliminated by the use of auto-interpretation computer software. In this stage, the combination of particular nucleic acid amplification products identified from the CE analysis of the PCR-SSP reactions (each of which indicates the presence of a particular HLA polymorphism), are interpreted automatically by the auto-interpretation software according to the invention.

Finally, the software provides an “Output” (typing result, bottom row) that indicates to which specific HLA genotype the nucleic acid sample belongs. In the prior art protocol, however, the user must subjectively interpret the results of the large number of separate PCR reactions to obtain a typing result.

The auto-interpretation software may be based, for example, on digital look-up tables, or any other suitable means of data comparison and interpretation.

All references cited herein are incorporated by reference in their entirety. Unless otherwise defined, all technical and scientific terms used herein have the same meaning as commonly understood by one of ordinary skill in the field to which this invention belongs. Unless otherwise indicated, the methods required for practising the invention are conventional techniques known to the person skilled in the art with knowledge of texts incorporated by reference (for example Sambrook J. et al. (2001), Molecular Cloning: a Laboratory Manual, Cold Spring Harbor Press, Cold Spring Harbor, N.Y., and other references listed herein).

### EXAMPLES

By way of example only, embodiments of the present invention will now be described in detail.

The invention provides improved methods and apparatus for determining the genotype of a sample of genetic material derived from a test subject or patient. More specifically, the invention provides a method for separating and detecting amplified nucleic acid (DNA) products from a sample and relating the presence of such amplification products to the presence of specific alleles within the sample, thus determining the genotype. The invention has particular application in major histocompatability complex (MHC) genotyping, and especially in human leukocyte antigen (HLA) typing.

Accordingly, the present invention provides for the identification of genotype of one or more HLA genes of an individual. In the population, the same gene may be subject to allelic variation due to polymorphisms in the gene. Thus, the methods, systems, kits and apparatus of the present invention are particularly directed towards the determination of which polymorphisms are present in the HLA genes under test and, thus, characterisation of the specific allele carried by the individual.

In accordance with the methods, systems, kits and apparatus of the invention, genotype of the HLA genes under analysis is suitably determined via primer extension and amplification based techniques, such as polymerase chain reaction (PCR). Short sequence-specific single-stranded oligonucleotide primers are used to amplify regions of the HLA genes in which sequence polymorphisms have been identified or are known to occur. The oligonucleotide primers are synthesised by techniques known in the art. Typically the regions of the HLA gene that are amplified by the primer extension reaction are short regions of less than 1 kbp in length, preferably less than 500 bp in length, and more preferably less than 300 bp in length. Inter alia, this allows faster throughput during the capillary electrophoresis analysis phase of the method of the invention.

A biological sample (e.g. a tissue sample) for use in accordance with the invention may be provided fresh ex vivo or may have been previously frozen, refrigerated or preserved in an appropriate preservative. Suitably, the sample is blood or cord blood, but it can be any tissue sample such as a nasal or cheek swab, saliva, urine, lymph fluid, semen, or a cell sample from a hair follicle or the skin. Ex vivo blood samples are suitably mixed with an anti-coagulant such as ethylenediaminetetraacetic acid (EDTA) or acid-citrate-dextrose (ACD).

Isolation of DNA from the biological sample can be achieved by a method suitably known in the art, for example by use of the BioGene-Expuze DNA isolation kits (Texas BioGene, Inc., Richardson, Tex., USA). By way of example, when DNA is isolated from blood: red blood cells are removed by lysis using a hypotonic solution, and then the remaining white cells are treated with detergent-containing solution to release DNA from the nuclei; the lysate is washed with buffers to remove contaminates and elute the DNA. Other simple and rapid methods of preparing samples for PCR could be employed and are described, for example, in Higuchi, 1989, in: PCR Technology: Principles and Applications for DNA Amplification, ed. H. A. Erlich. Stockton Press, New York, N.Y.:31-35. The isolated DNA can suitably comprise gene-derived sequences, including but not limited to genomic DNA and/or cDNA.

Primers provided for use in the DNA amplification reaction include forward and reverse primers. The primers are sequence-specific primers, directed towards allelic sequences that may be present in the template DNA. According to a preferred embodiment of the invention, the primers are directed towards alleles of the human MHC genes, i.e. the HLA genes. Sequence specific primers may be synthesised de novo, or they may be purchased from a supplier known in the field. Typically, the suitability of primers for use is assessed on the basis of a number of characteristics, including, for example: base composition; length; complementarity to potential priming sites on the template DNA; presence of repeated or self-complimentary sequences (which can form hairpin structures in use and prevent the oligonucleotide from annealing to the priming site on the template DNA); annealing temperature; and complementarity to other primers present in the DNA amplification reaction. Conveniently, a computer program or programs may be used to facilitate the design and selection of suitable primers.

Suitably, the amplification of DNA is achieved using an iterative DNA replication reaction such as a polymerase chain reaction (PCR). Each DNA amplification reaction may contain numerous rounds of DNA replication. Such a reaction is performed as would be appreciated by the person skilled in the art, and as described in detail in Sambrook et al., 2001. The amplification reaction is of the PCR-SSP type, and uses sequence-specific primers designed and selected as described above to amplify specific sequences from the template DNA. Typically, the DNA amplification reaction further comprises appropriate positive and/or negative controls, as described by Sambrook et al., pp 8.21.

Preferably, the DNA amplification reaction contains at least one set of sequence-specific primers, i.e. a “primer set”, as described herein before. In brief, a primer set is defined as a set of primers that is adapted to amplify target nucleic acid sequences associated with more than one specific polymorphism, and containing at least one forward primer and at least one reverse primer. More preferably, a primer set contains two specific primer pairs, and still more preferably, a primer set further comprises a positive control primer pair.

Thus, primer sets used in accordance with the invention are capable of directing multi-specific PCR (as described above), and in the presence of an appropriate nucleic acid sample, more than one polymorphic sequence (e.g. 2 polymorphic sequences) are amplified simultaneously. In this way, the invention enables simultaneous detection of numerous polymorphisms from a single amplification reaction. By providing primer sets adapted to enable multi-specific PCR, the number of DNA amplification reactions that is required to genotype a sample is reduced. Multi-specific PCR may even allow a specific allele or polymorphism within the template DNA (sample nucleic acid) to be amplified and detected even when the exact sequence is not known, for example, by the provision of a number of alternative forward and reverse primers in a single PCR-SSP reaction. Importantly, the invention enables genotyping of a number of different loci, for example a number of HLA alleles, to be achieved in one nucleic acid amplification reaction, by the inclusion of numerous sequence-specific primers.

The separation of DNA amplification products is preferably achieved by a method of capillary electrophoresis (CE). It will be appreciated by one skilled in the art that the term “capillary electrophoresis” describes any of a number of related separation techniques, as described above. Suitable practical methods of performing CE are described in Lubin, I. M., 1999. HFE Genotyping using allele specific polymerase chain reaction and capillary electrophoresis. Arch Pathol Lab Med., 123, pp 1177-1181. Typically, the separation process is automated, and uses multiple capillary channels (e.g. one capillary tube per PCR-SSP reaction), so that a number of concurrent separations are performed simultaneously.

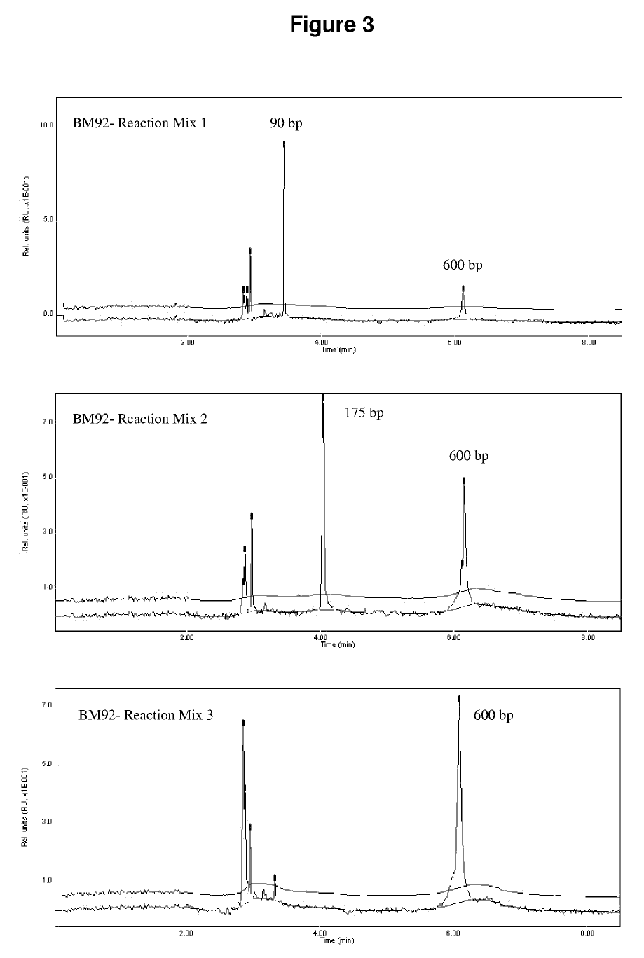

Preferably, the DNA amplification products are labelled with ethidium bromide (EtBr) to enable bands of DNA to be observed following separation. Suitably, EtBr is included in a buffer used during capillary electrophoresis. Detection of bands is preferably automated, suitably using an LED light source and an appropriate optical detector linked to a computer and appropriate software, such that the presence of bands can be digitally recorded, quantified and reported to the operator. Appropriate software may be bespoke. The molecular size of DNA amplification products present in each band is determined by comparison with known values from DNA reference markers included in the CE separation phase of the invention. Results may be presented in the form of at least one electropherogram, with bands represented by peaks on the electropherogram, for example, as shown in FIGS. 3 to 8. The results of slab gel electrophoresis separations have conventionally been resolved qualitatively by eye (see FIG. 9), so the quantitative digital detection of the present invention offers clear advantages in terms of speed and accuracy of obtaining results.

To enable the genotype of a sample to be determined, at least one typing table may be created (see Table 1) to allow the presence and absence of peaks on the electropherogram to be correlated with the presence and absence of particular sequences within the nucleic acid sample (or template DNA). The sequences are suitably HLA alleles. A typing table is formulated from what is known about the primer pairs and primer sets included in the DNA amplification reaction. For example, it may be known that a first set of sequence-specific primers will amplify a DNA product of 90 bps in length when a first HLA allele is present in the sample nucleic acid; however, if a second, alternative allele is present, no product is amplified, because the sequence polymorphism that is required for the binding of one or more of the sequence-specific primers is not present. By way of example, it may further be known that a second primer set will amplify a product of 150 base pairs in length when the above-mentioned second allele is present, but not when the above-mentioned first allele is present. Thus, by correlating one or more nucleic acid amplification products (following PCR-SSP using primer sets in accordance with the invention), with the presence of particular HLA polymorphisms, a matrix can be compiled in the form of a typing table. For illustration purposes only, a simple example of a typing table is given in Table 2, below.

TABLE 2
Example of a typing table

Reaction mix^{a} and expected
amplification product size^{b}

| | 1 | 2 |
|---|---|---|
| Allele | 90 bp | 150 bp |
| A-1 | + | |
| A-2 | | + |

^{a}each reaction mix contains a primer set
^{b}size (in base pairs) of amplification product expected according to the type of allele present in the template DNA

Suitably, empirical data regarding the presence and absence of particular amplification products gathered from sample template DNA can be compared to the typing table and the presence of certain HLA alleles determined. Typically, in accordance with the invention, the presence and absence of particular amplification products is determined by the presence and absence of peaks on one or more electropherograms following CE. Preferably, the comparison is made automatically using computer software. However, it is possible that the data resulting from CE is analysed manually by the operator. Suitably, therefore, the typing table is incorporated into analysis software, such that the operator is simply presented with a report of the determined genotype.

It will be appreciated that more than one typing table may be used, or may be required, to determine the genotype of a sample. Suitably, a typing table may also contain other information known in the art or derived empirically, for example information from serological or cellular assays. Such additional information can be suitably used to further verify the tissue typing results obtained according to the methods of the invention.

According to the invention, a kit is provided for the determination of the HLA genotype of a sample, comprising at least one sequence specific primer set directed towards one or more priming sites of any HLA allele. Typically, the kit comprises at least two or more primer sets such that more multiple HLA polymorphisms (and HLA alleles) can be detected or screened for. More preferably, the kit comprises 96 or less primer sets, to enable the genotyping of the HLA-A, HLA-B and HLA-DR genes simultaneously. In such an HLA-A, HLA-B and HLA-DR genotyping kit, the kit preferably comprises 48 primer sets. Similarly, a kit may be provided for simultaneously genotyping the HLA-A, HLA-B, HLA-C, HLA-DR and HLA-DQ genes. Likewise, a kit for the simultaneous genotyping of the HLA-A, HLA-B, HLA-C, HLA-DR and HLA-DQ genes comprises 96 or less primer sets; more preferably comprises 78 or less primer sets; and most preferably comprises 63 primer sets.

Suitably, the kits of the invention comprise multi-well plates. Preferably, the kits comprise 96- or 384-well plates, and most preferably 96-well plates. More preferably, each of the primer sets required for typing either: HLA-A, HLA-B, and HLA-DR genes; or the HLA-A, HLA-B, HLA-C, HLA-DR and HLA-DQ genes, are pre-aliquoted into separate wells of the multi-well plates. Most preferably, the primer sets are contained in each well in dried form for easy of storage and stability; for example, freeze dried or lyophilised.

The kits of the invention may further comprise suitable buffers and/or pipette tips and/or tubes for preparing and handling components for the PCR-SSP reactions. Typically, the kit further comprises capillary tubes and buffers, reagents and substrates suitable for CE. Preferably, the components of the kit are sterile. Suitably, the kit further comprises operating instructions in the form of a protocol for performing the method of the invention and appropriate typing tables.

According to the invention, a device is provided to analyse the products of the DNA amplification reaction. Suitably, the device comprises an LED light source and an appropriate optical detector linked to a computer and appropriate software, such that the presence of bands of separated PCR-SSP products can be recorded, quantified and reported digitally. The presence of bands is quantified according to the relative migration time, and the molecular size determined by comparison with known values from DNA reference markers. Results may be presented in the form of one or more electropherograms (see for example FIGS. 3 to 8). Preferably the device is adapted to separate the products of the DNA amplification reaction by a method of CE, most preferably, by CGE. Conveniently, the device may still further be adapted to perform the DNA amplification reaction to amplify specific sequences (polymorphisms) from template DNA. The device has particular application to the determination of HLA genotype. The device preferably comprises several electrophoresis capillaries for concurrent analysis of results, allowing automation for high-throughput applications.

Thus, the methods and apparatus of the present invention enable rapid and cost effective tissue typing of HLA genes in a sample, with advantages in terms of automation and reduction of running costs and human error.

The invention provides primer sets that are adapted to enable multi-specific PCR of target nucleic acids. In this way, the need to run numerous separate DNA amplification reactions (either concurrently or in series) is reduced or even eliminated. By way of example, typing of the HLA-A locus using only one set of primers at a time would require 24 separate DNA amplification reactions, each comprising multiple thermal cycles and subsequent detection of amplification products. However, by carrying out two specific amplifications per reaction only 12 separate reactions are needed. By adapting each primer set to detect four specific polymorphisms per reaction, only 6 separate reactions is required to establish the genotype of the HLA-A locus. Thus, by using multi-specific PCR-SSP, the extra time required and the demands placed on a human operator to run numerous separate reactions are reduced, as are the likelihood of incidences of cross-contamination and human error inherent in setting up a large number of separate reactions.

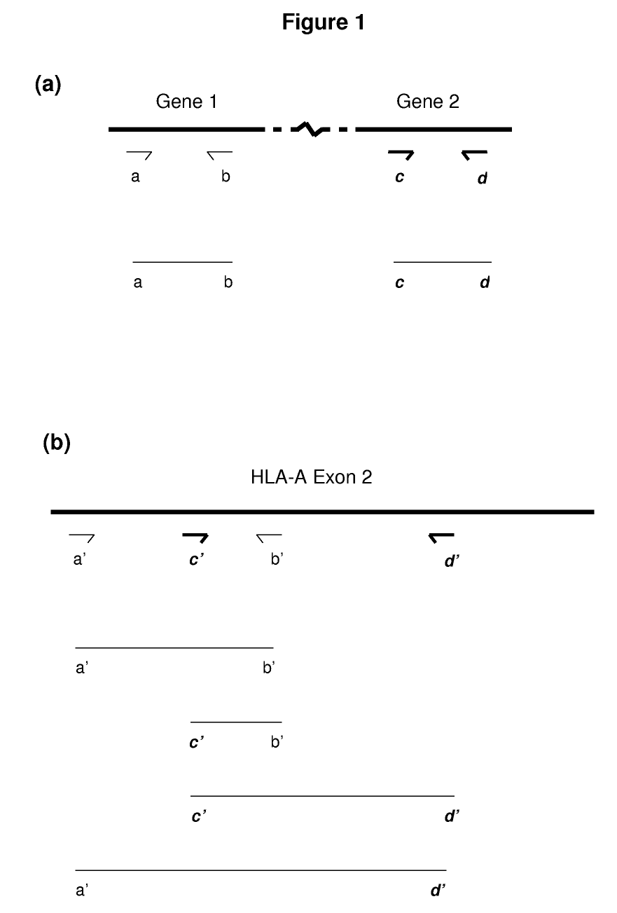

With reference to FIG. 1a, it will be appreciated that a primer sets of the invention may include two different specific primer pairs (a, b; and c, d), each of which targets a specific sequence at a locus distally situated on the template DNA in relation to the other (for example, the specific sequences may be located on different chromosomes, or in different genes of the same chromosome, or in different exons of the same gene separated by intervening introns). This can be considered to represent inter-loci multi-specific PCR. In contrast, with reference to FIG. 1b, the two specific primer sets (a′, b′; and c′, d′), may target sequences that are proximally located with respect to each other (i.e. within the same exon), and the target sequences could even overlap, as depicted. This example can be considered to represent intra-locus multi-specific PCR. Due to the close proximity and/or overlapping nature of the target sequences, intra-locus multi-specific DNA amplification can yield additional DNA amplification products by cross-amplification. For example, two sets of primers can give rise to four amplification products, as shown in FIG. 1b. In this case, the interpretation means must be adapted to recognise and account for this eventuality. Hence, the present invention also addresses the need for rapid and accurate separation and identification of DNA amplification products from a multiplex amplification reaction by providing capillary electrophoresis (CE)-based separation and digital analysis techniques. It is important to note, that particularly in the case of intra-locus multi-specific PCR, where there is a possibility of cross-amplification causing several nucleic acid products, it may not be possible to resolve those product using convention means of separation and analysis, such as slab gel electrophoresis and manual interpretation, e.g. by eye.

The advantages of employing CE in place of other separation techniques, such as agarose gel electrophoresis, are considerable, and have been described hereinbefore. Hence, according to the present invention, the combination of CE with primer sets that enable multi-specific DNA amplification reactions provide rapid and automated resolution and detection of DNA amplification products and, hence, effective and rapid genotyping. In particular, the use of automated CE reduces the burden on the skilled operator compared to traditional gel separation and, coupled with savings in terms of time and cost, there are significant advantages in the sensitivity/accuracy of product detection and the reduction of human error. For example, the increased accuracy of digital detection of DNA bands following nucleic acid separation compared to resolving bands and relative migration distances by eye is important in making a swift and accurate determination of genotype.

The benefits described herein are of particular importance in HLA typing, when determination of the HLA genotype may be time sensitive. For example, HLA typing of organs and tissues provided for transplant is extremely time critical, because the recipient's life may depend upon receiving compatible transplant tissue as quickly as possible. Therefore accurate, fast HLA typing as provided by the present invention is essential.

It will be appreciated that the invention may also be applied to MHC genotyping in species other than human. For example, the invention may enable MHC typing of animal tissues in veterinary surgery prior to allograft transplant (between animals of the same species) or xenograft transplant (between animals and humans, or between animals of different species). By way of example, the invention may be applied to SLA typing of pig-derived organs, tissues and/or bone marrow.

The invention may also be used to genotype other DNA sources, including for example viruses, bacteria, other pathogenic organisms, viral-infected host cells, or cancer cells.

The invention is further illustrated and exemplified by the following non-limiting examples.

### Example 1

### Typing of a Specific HLA-A Allele, A*25, using Separate DNA Amplification Reactions to Amplify Targets at the Same Locus

Template DNA Preparation

Genomic DNA was isolated from the human B-lymphoblastoid cell lines BM92 and ISH4. These cell lines with known HLA genotypes were obtained from IHWG Cell and Gene Bank. The BM92 cell line is homozygous for the HLA-A*2501 allele; the ISH4 cell line is heterozygous for A*0218 and A*1101. DNA isolation was performed using the BioGene-Expuze DNA isolation kit (Texas BioGene, Inc., Richardson, Tex., USA). The absorbance ratio at A260/A280 was determined, and was greater than 1.65. The DNA preparation was checked by agarose gel electrophoresis and showed a single band of size greater than 10 kb. The concentration of DNA was between 10-80 ng/μl.

The following amplification, separation and analysis steps were performed for DNA from each cell line.

PCR Preparation

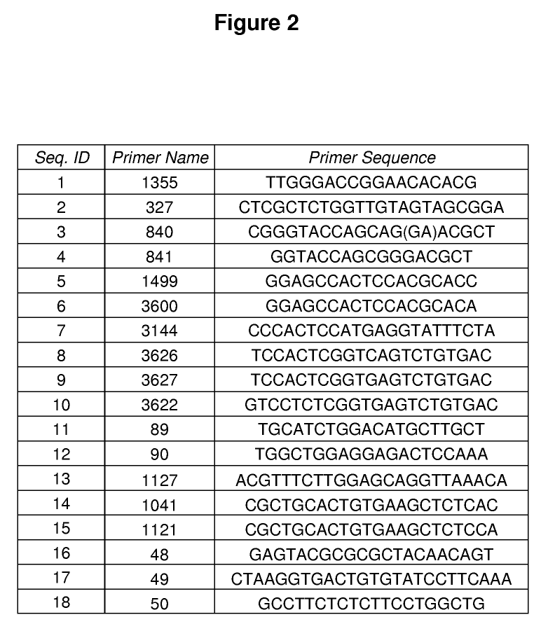

The master PCR mix was prepared by addition of 4.5 μl of Taq polymerase enzyme (at 5 U/μl) to 8M buffer (75 mM Tris-HCl, 50 mM KCl, 0.08% Nonidet P40, 1.5 mM MgCl2, 0.2 mM of dNTP). To the master mix solution was added 204 μl of prepared template DNA, and the solution vortexed to mix well. 8 μl of master mix was added to each of three wells in a multi-well plate, and the plate sealed with a thermal cycler reaction plate sealer. Three reaction mixes were prepared containing separate sets of sequence specific primers to amplify different target sequences of the template DNA. Reaction Mix 1 contained primers 1355 and 327 (according to SEQ. ID NOs 1 and 2, respectively; see FIG. 2), and was added to the first well in the plate; Reaction Mix 2 contained primers 840, 841, 1499 and 3600 (according to SEQ. ID NOs 3, 4, 5 and 6), and was added to the second well; Reaction Mix 3 contained primers 3144, 3626, 3627 and 3622 (according to SEQ. ID NOs 7, 8, 9 and 10), and was added to the third well. In addition, primers 89 and 90 (according to SEQ. ID NOs 11 and 12) were added to each well as positive controls for the DNA amplification reaction (primers 89 and 90 amplify a housekeeping gene present in the template DNA resulting in a detectable amplification product with a size of 600 bp). The plate was placed into a 96-well thermal cycler and a DNA amplification reaction performed as described in the following section.

DNA Amplification Reaction

Three DNA amplification reactions, one per well, were run according to the program shown in Table 3 using a GeneAmp 9600 or 9700 thermal cycler (Perkin Elmer, Boston, Mass., USA). Total reaction time was about 1 hour 25 minutes.

TABLE 3
DNA amplification program for Example 1

| Segment | Cycle number | Temperature | Time | |
|---|---|---|---|---|
| 1 | 1 | 96° C. | 2.5 | min |
| 2 | 10 | 96° C. | 15 | sec |
| | | 65° C. | 60 | sec |
| 3 | 22 | 96° C. | 15 | sec |
| | | 62° C. | 50 | sec |
| | | 72° C. | 30 | sec |

| | | | |
|---|---|---|---|
| 4 | 1 | 4° C. | Until removed |

Separation and Detection of DNA Amplification Products

The PCR amplification products from each of the three wells were separated by capillary electrophoresis using the HDA-GT12 multi-capillary system (eGene Inc., Irvine, Calif., USA). Samples were injected at 5 kV for 20 seconds, and then separation performed at 5 kV for 500 seconds. Three CE separation reactions were performed in total, one per well.

Data Analysis

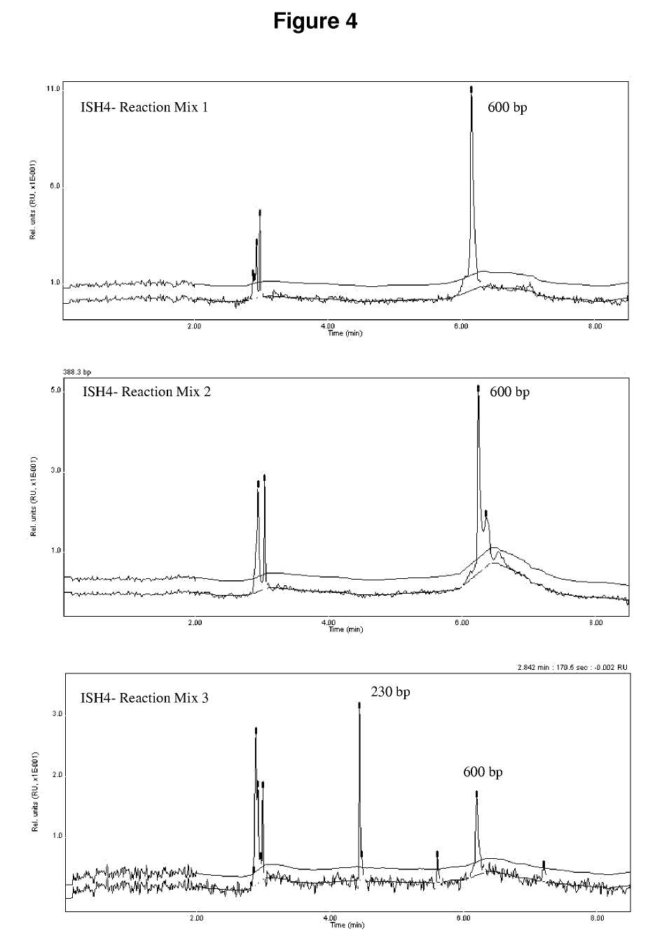

EtBr-stained bands produced following CE separation were detected using an LED light source, and results returned to the operator digitally by BioCalculator Software (eGene Inc., Irvine, Calif., USA). Results were presented in the form of an electropherogram, with separate electropherograms for each of the three separations. Electropherograms for the BM92 and ISH4 cell lines are shown in FIGS. 3 and 4, respectively. The molecular size of DNA molecules in the bands was calculated based on the relative migration time compared to standard DNA reference markers included in the CE separation technique.

Determination of HLA Genotype

A typing table was created based upon the primer sets used in each reaction mix and the amplification products expected from each reaction mix following its use in typing the following HLA-A alleles: HLA-A*2501/03/04 and HLA-A*2502. The grouping of HLA-A*2501/03/04 indicates that the actual HLA-A*25 allele present may be any of those three. The typing table is given in Table 4.

TABLE 4
Typing table for Example 1

| | | Reaction mix and expected | | |
|---|---|---|---|---|
| | | amplification product size | | |
| Serology | | 1 | 2 | 3 |
| type | Allele | 90 bp | 175 bp | 230 bp |
| A25 | A*2501/03/04 | + | + | |
| A25 | A*2502 | + | + | + |

Referring to FIG. 3, the three electropherograms illustrate that BM92 template DNA yielded a 90 base pair (bp) amplification product from reaction mix 1 and a 175 bp product from reaction mix 2, but did not yield a 230 bp product from reaction mix 3. The 600 bp positive control is also visible in each electropherogram, illustrating successful DNA amplification and separation and detection of products in each case. Hence, according to the typing table the HLA-A*25 allele present in the BM92 cell line is one of A*2501/03/04, and is not A*2502, which is consistent with the known genotype.

Referring to FIG. 4, aside from the 600 bp positive control the ISH4 template DNA showed only a 230 bp product from reaction mix 3. Hence, according to the typing table the ISH4 cell line does not have any of the assayed HLA-A*25 allele types, which is consistent with the known genotype.

### Example 2

### Typing of a Specific HLA-A Allele, A*25, using a Single Multi-Specific DNA Amplification Reaction to Amplify Targets at the Same Locus

Template DNA Preparation

Genomic DNA was isolated from the human B-lymphoblastoid cell lines BM92 and ISH4. These cell lines with known HLA genotypes were obtained from IHWG Cell and Gene Bank. The BM92 cell line is homozygous for the HLA-A*2501 allele; the ISH4 cell line is heterozygous for A*0218 and A*1101. DNA isolation was performed using the BioGene-Expuze DNA isolation kit (Texas BioGene, Inc., Richardson, Tex., USA). The absorbance ratio at A260/A280 was determined, and was greater than 1.65. The DNA preparation was checked by agarose gel electrophoresis and showed a single band of size greater than 10 kb. The concentration of DNA was between 10-80 ng/μl.

The following amplification, separation and analysis steps were performed for DNA from each cell line.

PCR Preparation

The master PCR mix was prepared by addition of 4.5 μl of Taq polymerase enzyme (at 5U/μl) to 8M buffer (75 mM Tris-HCl, 50 mM KCl, 0.08% Nonidet P40, 1.5 mM MgCl2, 0.2 mM of dNTP). To the master mix solution was added 204 μl of prepared template DNA, and the solution vortexed to mix well. 8 μl of master mix was added to a single well in a multi-well plate, and the plate sealed with a thermal cycler reaction plate sealer. A reaction mix was prepared containing the following sequence-specific primers to amplify target sequences of the template DNA: primers 1355, 327, 840, 841, 1499, 3600, 3144, 3626, 3627 and 3622 (according to SEQ. ID NOs 1 to 10). In addition, primers 89 and 90 (according to SEQ. ID NOs 11 and 12) were added as positive controls for the DNA amplification reaction (primers 89 and 90 amplify a housekeeping gene present in the template DNA resulting in a detectable amplification product with a size of 600 bp). This combination of sequence-specific primers can be considered to represent a single “primer set”. The reaction mix was added to the well in the plate. The plate was placed into a 96-well thermal cycler and a DNA amplification reaction performed as described in the following section.

DNA Amplification Reaction

A DNA amplification reaction was run according to the program shown in Table 5 using a GeneAmp 9600 or 9700 thermal cycler (Perkin Elmer, Boston, Mass., USA). Total reaction time was about 1 hour 25 minutes.

TABLE 5
DNA amplification program for Example 2

| Segment | Cycle number | Temperature | Time | |
|---|---|---|---|---|
| 1 | 1 | 96° C. | 2.5 | min |
| 2 | 10 | 96° C. | 15 | sec |
| | | 65° C. | 60 | sec |
| 3 | 22 | 96° C. | 15 | sec |
| | | 62° C. | 50 | sec |
| | | 72° C. | 30 | sec |

| | | | |
|---|---|---|---|
| 4 | 1 | 4° C. | Until removed |

Separation and Detection of DNA Amplification Products

PCR amplification products were separated by capillary electrophoresis using the HDA-GT12 multi-capillary system (eGene Inc., Irvine, Calif., USA). Samples were injected at 5 kV for 20 seconds, and then separation performed at 5 kV for 500 seconds.

Data Analysis

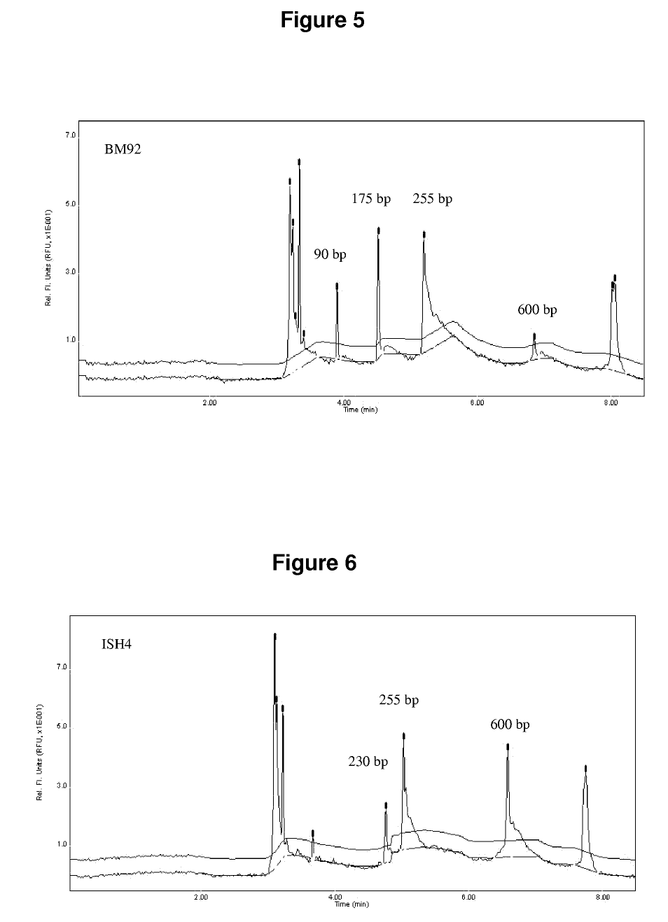

EtBr-stained bands produced following CE separation were detected using an LED light source, and results returned to the operator digitally by BioCalculator Software (eGene Inc., Irvine, Calif., USA). Results were presented in the form of an electropherogram. Electropherograms for the BM92 and ISH4 cell lines are shown in FIGS. 5 and 6, respectively. The molecular size of DNA molecules in the bands was calculated based on the relative migration time compared to standard DNA reference markers included in the CE separation technique.

Determination of HLA Genotype

A typing table was created based upon the primer sets used in the multiplexing reaction mix and the amplification products expected following its use in typing the following HLA-A alleles: HLA-A*2501/03/04 and HLA-A*2502. The grouping of HLA-A*2501/03/04 indicates that the actual HLA-A*25 allele present may be any of those three. The typing table is given in Table 6.

TABLE 6
Typing table for Example 2

| | | Reaction mix and expected | | | |
|---|---|---|---|---|---|
| | | amplification product size | | | |
| Serology | | 1 | | | |
| type | Allele | 90 bp | 175 bp | 230 bp | 255 bp |
| A*25 | A*2501/03/04 | + | + | | + |
| A*25 | A*2502 | + | + | + | + |

Referring to FIG. 5, the electropherogram illustrates that BM92 template DNA yielded a 90 bp, 175 bp and 255 bp amplification products, but did not show a 230 bp product. The 255 bp product is an additional amplification product resulting from cross-amplification resulting from a particular combination of primers in the primer set in the multi-specific DNA amplification reaction. The 600 bp positive control is visible, illustrating successful DNA amplification and separation and detection of products in each case. Hence, according to the typing table the HLA-A*25 allele present in the BM92 cell line is one of A*2501/03/04, and is not A*2502, which is consistent with the known genotype.

Referring to FIG. 6, aside from the 600 bp positive control the ISH4 template DNA showed only 230 bp and 255 bp products, but not 90 bp or 175 bp products. Hence, according to the typing table the ISH4 cell line does not have any of the assayed HLA-A*25 allele types, which is consistent with the known genotype.

### Example 3

### Typing of Two HLA Alleles, A*25 and DR*04, using Multi-Specific DNA Amplification Reactions to Amplify Targets at Different Loci

Template DNA Preparation

Genomic DNA was isolated from the human B-lymphoblastoid cell line BM92. This cell line has a known HLA genotype and was obtained from IHWG Cell and Gene Bank. The BM92 cell line is homozygous for the HLA-A*2501 and HLA-DR*0404 alleles. DNA isolation was performed using the BioGene-Expuze DNA isolation kit (Texas BioGene, Inc., Richardson, Tex., USA). The absorbance ratio at A260/A280 was determined, and was greater than 1.65. The DNA preparation was checked by agarose gel electrophoresis and showed a single band of size greater than 10 kb. The concentration of DNA was between 10-80 ng/μl.

The following amplification, separation and analysis steps were performed for DNA from each cell line.

PCR Preparation

The master PCR mix was prepared by addition of 4.5 μl of Taq polymerase enzyme (at 5U/μl) to 8M buffer (75 mM Tris-HCl, 50 mM KCl, 0.08% Nonidet P40, 1.5 mM MgCl2, 0.2 mM of dNTP). To the master mix solution was added 204 μl of prepared template DNA, and the solution vortexed to mix well. 8 μl of master mix was added to each of five wells in a multi-well plate, and the plate sealed with a thermal cycler reaction plate sealer. Three reaction mixes were prepared containing separate sets of sequence specific primers (i.e. primer sets) to amplify different target sequences of the template DNA. Reaction Mix 1 contained primers 1355 and 327 (according to SEQ. ID NOs 1 and 2, respectively; see FIG. 2), and was added to the first well in the plate; Reaction Mix 2 contained primers 840, 841, 1499 and 3600 (according to SEQ. ID NOs 3, 4, 5 and 6), and was added to the second well; Reaction Mix 3 contained primers 1127, 1041 and 1121 (according to SEQ. ID NOs 13, 14 and 15), and was added to the third well. Reaction Mix 4 contained primers 48, 49 and 50 (according to SEQ. ID NOs 16, 17, and 18), and was added to the fourth well. Reaction Mix 5 contained primers 1355, 327, 840, 841,1499, 3600,1127,1041,1121, 48, 49 and 50 (according to SEQ. ID NOs 1 to 6 and 13 to 18), and was added to the fifth well. In addition, primers 89 and 90 (according to SEQ. ID NOs 11 and 12) were added to each well as positive controls for the DNA amplification reaction (primers 89 and 90 amplify a housekeeping gene present in the template DNA resulting in a detectable amplification product with a size of 600 bp). The plate was placed into a 96-well thermal cycler and a DNA amplification reaction performed as described in the following section.

In the above experiment, Reaction Mix 1 is a multiplex PCR, because is contains a specific primer pair (SEQ. ID NOs 1 and 2) and a control primer pair (SEQ. ID NOs 11 and 12), but unlike Reaction Mixes 2 to 5, it does not enable multi-specific PCR, since only one specific nucleic acid amplification product can be produced.

DNA Amplification Reaction

Five DNA amplification reactions, one per well, were run according to the program shown in Table 7 using a GeneAmp 9600 or 9700 thermal cycler (Perkin Elmer, Boston, Mass., USA). Total reaction time was about 1 hour 25 minutes.

TABLE 7
DNA amplification program for Example 3

| Segment | Cycle number | Temperature | Time | |
|---|---|---|---|---|
| 1 | 1 | 96° C. | 2.5 | min |
| 2 | 10 | 96° C. | 15 | sec |
| | | 65° C. | 60 | sec |
| 3 | 22 | 96° C. | 15 | sec |
| | | 62° C. | 50 | sec |
| | | 72° C. | 30 | sec |

| | | | |
|---|---|---|---|
| 4 | 1 | 4° C. | Until removed |

Separation and Detection of DNA Amplification Products

The PCR amplification products from each of the five wells were separated by capillary electrophoresis using the HDA-GT12 multi-capillary system (eGene Inc., Irvine, Calif., USA). Samples were injected at 5 kV for 20 seconds, and then separation performed at 5 kV for 500 seconds. Five CE separation reactions were performed in total, one per well.

Data Analysis

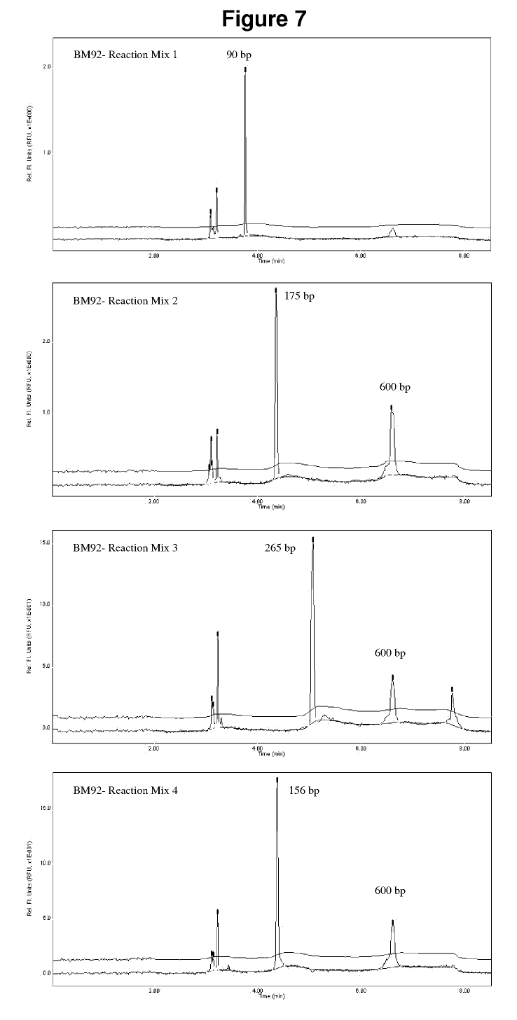

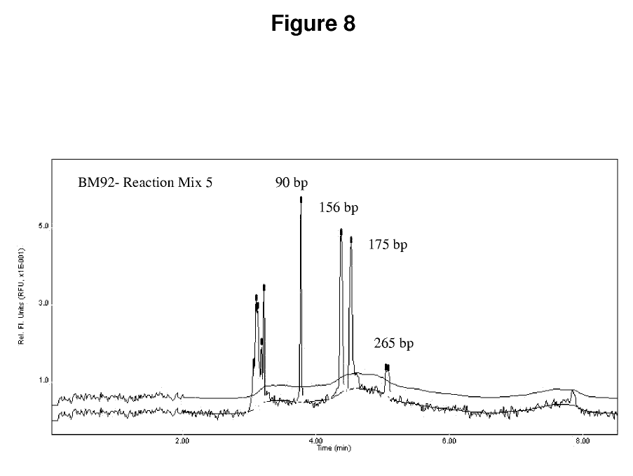

EtBr-stained bands produced following CE separation were detected using an LED light source, and results returned to the operator digitally by BioCalculator Software (eGene Inc., Irvine, Calif., USA). Results were presented in the form of an electropherogram, with separate electropherograms for each of the five separations. Electropherograms for the first four wells (containing reaction mixes 1 to 4) are shown in FIG. 7; an electropherogram for the fifth well (containing reaction mix 5) is shown in FIG. 8. The molecular size of DNA molecules in the bands was calculated based on the relative migration time compared to standard DNA reference markers included in the CE separation technique.

Determination of HLA Genotype

A typing table was created based upon the primer sets used in each reaction mix and the amplification products expected from each reaction mix following its use in typing the following HLA-A and HLA-DRB1 alleles: HLA-A*2501-2504 and HLA-DRB1*0401-44. The grouping of HLA-A*2501-2504 indicates that the actual HLA-A*25 allele present may be any of those four. The grouping of HLA-DRB1*0401-44 indicates that the actual DRB1*04 allele present may be any of those forty-four. The typing table is given in Table 8.

TABLE 8
Typing table for Example 3

| | | Reaction mix and expected amplification product size | | | | | | | |
|---|---|---|---|---|---|---|---|---|---|
| Serology | | 1 | 2 | 3 | 4 | 5 | | | |
| type | Allele | 90 bp | 175 bp | 265 bp | 156 bp | 90 bp | 156 bp | 175 bp | 265 bp |
| A25 | A*2501-04 | + | + | | | + | | + | |
| DR4 | DRB1*0401-44 | | | + | + | | + | | + |

Referring to FIG. 7, the four electropherograms illustrate that BM92 template DNA yielded a 90 base pair (bp) amplification product from reaction mix 1 and a 175 bp product from reaction mix 2; hence, according to the typing table the HLA-A*25 allele present in the BM92 cell line is one of A*2501-04, which is consistent with the known genotype. In addition, the template DNA yielded a 265 base pair (bp) amplification product from reaction mix 3 and a 156 bp product from reaction mix 4; hence, according to the typing table the HLA-DR*4 allele present in the BM92 cell line is one of DRB1*0401-44, also which is consistent with the known genotype.

Reaction mix 5 was a multi-specific PCR reaction in which the primer set was designed to target different HLA loci, i.e. targeting the HLA-A and HLA-DR loci (inter-loci multi-specific PCR). Referring to FIG. 8, the BM92 template DNA yielded products with sizes of 90, 156, 175 and 265 bp. Hence, according to the typing table the cell line has HLA allele types A*2501-04 and DRB1*04-01, which is consistent with the known genotype.

Although particular embodiments of the invention have been disclosed herein in detail, this has been done by way of example and for the purposes of illustration only. The aforementioned embodiments are not intended to be limiting with respect to the scope of the appended claims, which follow. Use of the techniques of DNA amplification and capillary electrophoresis is believed to be a routine matter for the person of skill in the art with knowledge of the presently described embodiments. It is contemplated by the inventors that various substitutions, alterations, and modifications may be made to the invention without departing from the spirit and scope of the invention as defined by the claims.

1. An in vitro method for determining the human leukocyte antigen (HLA) genotype of a nucleic acid sample, comprising the steps of:
providing at least one oligonucleotide primer set;
contacting the nucleic acid sample with the at least one primer set and subjecting the nucleic acid sample and at least one primer set to a nucleic acid amplification reaction;
determining the size of any nucleic acid amplification products produced in the nucleic acid amplification reaction; and
correlating the presence and/or absence of specific amplification products with the presence and/or absence of specific sequence polymorphisms in the nucleic acid sample;
wherein at least one of the at least one primer sets is a multi-specific primer set comprising at least one sequence-specific forward primer and at least one sequence-specific reverse primer and being adapted to amplify, in a nucleic acid amplification reaction, two or more specific target sequences that may be present in the nucleic acid sample, and wherein each of the specific target sequences comprises a sequence polymorphism that is known to be associated with an HLA allele and which may be present in the nucleic acid sample to be genotyped.

2. The method of claim 1, wherein the at least one sequence-specific forward primer and the at least one sequence-specific reverse primer of the multi-specific primer set constitute at least two specific primer pairs, each specific primer pair comprising a forward primer and a reverse primer and being adapted to amplify a specific target sequence that may be present in the nucleic acid sample.

3. The method of claim 2, wherein the forward primer and/or the reverse primer of each specific primer pair is complementary to a specific sequence polymorphism that may be present in the nucleic acid sample to be genotyped, and wherein each of the specific primer pairs produces a specific amplification product only in the presence of the specific sequence polymorphism.

4. The method of claim 1, wherein at least one multi-specific primer set comprises two specific primer pairs, each specific primer pair being adapted to amplify a specific target sequence that may be present in the nucleic acid sample, and wherein each of the specific target sequences is in a different genetic locus of the nucleic acid sample.

5. The method of claim 1, wherein at least one multi-specific primer set comprises two specific primer pairs, each specific primer pair being adapted to amplify a specific target sequence that may be present in the nucleic acid sample, and wherein each of the specific target sequences the same genetic locus of the nucleic acid sample.

6. The method of claim 1, wherein at least one multi-specific primer set comprises two specific primer pairs, each specific primer pair being adapted to amplify a specific target sequence that may be present in the nucleic acid sample, and wherein each of the specific target sequences the same genetic locus of the nucleic acid sample, and wherein the target sequences overlap.

7. The method of claim 1, wherein each of the primer sets further comprises a positive control primer pair, the control primer pair comprising a forward primer and a reverse primer and which is adapted to amplify a control sequence known to be present in the nucleic acid sample, and which control sequence is in a different gene or genes to those to be genotyped.

8. The method of claim 1, wherein at least one of the primer sets comprises one or more primers selected from the group comprising SEQ ID NOS. 1 to 18.

9. The method of claim 1, wherein each of the specific target sequences comprises at least one sequence polymorphism, the polymorphism being selected from the group comprising: single nucleotide polymorphisms (SNPs); insertions, substitutions and deletions of one or more nucleotides; and repetitive sequences (for example, microsatellites or repeats).

10. The method of claim 1, wherein the nucleic acid sample comprises genomic DNA (gDNA) or cloned DNA (cDNA).

11. The method of claim 1, wherein the nucleic acid sample is gDNA, and wherein the gDNA has previously been extracted from a biological sample.

12. The method of claim 1, wherein the biological sample is selected from the group consisting of epithelial tissue, blood, saliva, urine, semen, bone marrow, nasal fluid or tissue, and a hair follicle.

13. The method of claim 1, wherein the step of determining the size of any nucleic acid amplification products comprises separating the nucleic acid amplification products generated in each nucleic acid amplification reaction using a capillary electrophoresis (CE) separation technique.

14. The method of claim 13, wherein the CE separation technique is selected from the group consisting of capillary zone electrophoresis (CZE), capillary gel electrophoresis (CGE), capillary isoelectric focusing (CIEF), isotachophoresis (ITP), electrokinetic chromatography (EKC), micellar electrokinetic capillary chromatography (MECC or MEKC), micro emulsion electrokinetic chromatography (MEEKC), non-aqueous capillary electrophoresis (NACE) and capillary electrochromatography.

15. The method of claim 13, wherein the CE technique is automated.

16. The method of claim 1, wherein the step of correlating the presence and/or absence of specific amplification products with the presence and/or absence of specific sequence polymorphisms in the nucleic acid sample is carried out in a computer using auto-interpretation software, and wherein the software provides an output that reports the genotype information derived on the basis of the presence and/or absence of the specific sequence polymorphisms.

17. The method of claim 1, wherein the nucleic acid amplification reaction is the polymerase chain reaction (PCR).

18. The method of claim 1, wherein the at least one oligonucleotide primer sets are adapted to identify a specific allele of the HLA-A, HLA-B and HLA-DR genes.

19. The method of claim 1, wherein the at least one oligonucleotide primer sets are adapted to identify a specific allele of the HLA-A, HLA-B, HLA-C, HLA-DR and HLA-DQ genes.

20. The method of claim 1, wherein no more than 96 primer sets are provided.

21. The method of claim 1, wherein between 48 and 96 primer sets are provided.

22. The method of claim 1, wherein the step of contacting the nucleic acid sample with the at least one primer set and subjecting the nucleic acid sample and at least one primer set to a nucleic acid amplification reaction is performed in an array.

23. An in vitro method for determining the human leukocyte antigen (HLA) genotype of a nucleic acid sample that has been obtained from a biological sample, comprising the steps of:
providing at least one oligonucleotide primer set;
contacting the nucleic acid sample with each of the primer sets and subjecting the nucleic acid sample and each primer set to a nucleic acid amplification reaction;
separating any nucleic acid amplification products produced in each of the nucleic acid amplification reactions using a capillary electrophoresis (CE) separation technique;
determining the size of the amplification products that have been separated using CE, and correlating the presence and/or absence of specific amplification products with the presence and/or absence of specific sequence polymorphisms associated with HLA alleles in the nucleic acid sample; and
assigning an HLA genotype on the basis of the information derived from the presence and/or absence of the specific sequence polymorphisms associated with HLA alleles in the nucleic acid sample;
wherein at least one of the at least one primer sets is a multi-specific primer set comprising at least one sequence-specific forward primer and at least one sequence-specific reverse primer and being adapted to amplify, in a nucleic acid amplification reaction, two or more specific target sequences that may be present in the nucleic acid sample, and wherein each of the specific target sequences comprises a sequence polymorphism that is known to be associated with an HLA allele and which may be present in the nucleic acid sample to be genotyped.

24. The method of claim 23, wherein at least one of the multi-specific primer sets is adapted to amplify two specific target sequences located in different genetic loci.

25. The method of claim 23, wherein at least one of the multi-specific primer sets is adapted to amplify two specific target sequences located within the same genetic locus.

26. The method of claim 23, wherein at least one of the primer sets comprises one or more primers selected from the group comprising SEQ ID NOS. 1 to 18.

27. The method of claim 23, wherein the CE separation technique is selected from the group consisting of capillary zone electrophoresis (CZE), capillary gel electrophoresis (CGE), capillary isoelectric focusing (CIEF), isotachophoresis (ITP), electrokinetic chromatography (EKC), micellar electrokinetic capillary chromatography (MECC or MEKC), micro emulsion electrokinetic chromatography (MEEKC), non-aqueous capillary electrophoresis (NACE) and capillary electrochromatography.

28. The method of claim 23, wherein the at least one oligonucleotide primer sets are adapted to identify a specific allele of the HLA-A, HLA-B and HLA-DR genes.

29. The method of claim 23, wherein the at least one oligonucleotide primer sets are adapted to identify a specific allele of the HLA-A, HLA-B, HLA-C, HLA-DR and HLA-DQ genes.

30. The method of claim 23, wherein no more than 96 primer sets are required to identify the specific allele.

31. The method of claim 23, wherein between 48 and 96 primer sets are required to identify the specific allele.

32. The method of claim 23, wherein the step of contacting the nucleic acid sample with the at least one primer set and subjecting the nucleic acid sample and at least one primer set to a nucleic acid amplification reaction is performed in an array.

33. An in vitro method for determining the human leukocyte antigen (HLA) genotype of a nucleic acid sample that has been obtained from a biological sample, comprising the steps of:
(i) providing at least one oligonucleotide primer set;
(ii) contacting the nucleic acid sample with each of the primer sets and subjecting the nucleic acid sample and each primer set to a nucleic acid amplification reaction;
(iii) separating any nucleic acid amplification products produced in each of the nucleic acid amplification reactions using a capillary electrophoresis (CE) separation technique;
(iv) determining the size of the amplification products that have been separated using CE, and correlating the presence and/or absence of specific amplification products with the presence and/or absence of specific sequence polymorphisms associated with HLA alleles in the nucleic acid sample; and
(v) assigning an HLA genotype on the basis of the information derived from the presence and/or absence of the specific sequence polymorphisms associated with HLA alleles in the nucleic acid sample;
wherein at least one of the at least one primer sets is a multi-specific primer set comprising at least one sequence-specific forward primer and at least one sequence-specific reverse primer and being adapted to amplify, in a nucleic acid amplification reaction, two or more specific target sequences that may be present in the nucleic acid sample, and wherein each of the specific target sequences comprises a sequence polymorphism that is known to be associated with an HLA allele and which may be present in the nucleic acid sample to be genotyped;
and wherein steps (iv) and (v) are carried out using an auto-interpretation software program run on a computer, which auto-interpretation software program avoids the requirement for manual interpretation of data to assign an HLA genotype.

34. The method of claim 33, wherein at least one of the multi-specific primer sets is adapted to amplify two specific target sequences located in different genetic loci.

35. The method of claim 33, wherein at least one of the multi-specific primer sets is adapted to amplify two specific target sequences located within the same genetic locus.

36. The method of claim 33, wherein each of the primer sets further comprises a positive control primer pair, the control primer pair comprising a forward primer and a reverse primer and which is adapted to amplify a control sequence known to be present in the nucleic acid sample, and which control sequence is in a different gene or genes to those to be genotyped.

37. The method of claim 33, wherein the nucleic acid amplification reaction is the polymerase chain reaction (PCR).

38. The method of claim 33, wherein the at least one oligonucleotide primer sets are adapted to identify a specific allele of the HLA-A, HLA-B and HLA-DR genes.

39. The method of claim 33, wherein the at least one oligonucleotide primer sets are adapted to identify a specific allele of the HLA-A, HLA-B, HLA-C, HLA-DR and HLA-DQ genes.

40. The method of claim 33, wherein no more than 96 primer sets are required to identify the specific allele.

41. The method of claim 33, wherein between 48 and 96 primer sets are required to identify the specific allele.

42. The method of claim 33, wherein the step of contacting the nucleic acid sample with each of the primer sets and subjecting the nucleic acid sample and each primer set to a nucleic acid amplification reaction is performed in an array.

43. An in vitro method for determining the human leukocyte antigen (HLA) genotype of a nucleic acid sample that has been obtained from a biological sample, comprising the steps of:
providing at least one oligonucleotide primer set;
contacting the nucleic acid sample with the at least one primer set and subjecting the nucleic acid sample and at least one primer set to a nucleic acid amplification reaction;
separating any nucleic acid amplification products produced in the nucleic acid amplification reaction using a capillary electrophoresis (CE) separation technique;
determining the size of the amplification products that have been separated using CE, and correlating the presence and/or absence of specific amplification products with the presence and/or absence of specific sequence polymorphisms associated with HLA alleles in the nucleic acid sample; and
assigning an HLA genotype on the basis of the information derived from the presence and/or absence of the specific sequence polymorphisms associated with HLA alleles in the nucleic acid sample;
wherein each primer set comprises at least one sequence-specific forward primer and at least one sequence-specific reverse primer and is adapted to amplify, in a nucleic acid amplification reaction, two or more target sequences that may be present in the nucleic acid sample, and wherein at least one of the target sequences comprises a specific sequence polymorphism that is known to be associated with an HLA allele, and which may be present in the nucleic acid sample to be genotyped.

44. The method of claim 43, wherein at least one of the primer sets comprises one or more primers selected from the group comprising SEQ ID NOS. 1 to 18.

45. The method of claim 43, wherein the specific sequence polymorphism is selected from the group comprising: single nucleotide polymorphisms (SNPs); insertions, substitutions and deletions of one or more nucleotides; and repetitive sequences (for example, microsatellites or repeats).

46. The method of claim 43, wherein the CE separation technique is selected from the group consisting of capillary zone electrophoresis (CZE), capillary gel electrophoresis (CGE), capillary isoelectric focusing (CIEF), isotachophoresis (ITP), electrokinetic chromatography (EKC), micellar electrokinetic capillary chromatography (MECC or MEKC), micro emulsion electrokinetic chromatography (MEEKC), non-aqueous capillary electrophoresis (NACE) and capillary electrochromatography.

47. The method of claim 43, wherein the step of correlating the presence and/or absence of specific amplification products with the presence and/or absence of specific sequence polymorphisms associated with HLA alleles in the nucleic acid sample, is carried out in a computer using auto-interpretation software, and wherein the software provides an output that reports the genotype information derived on the basis of the presence and/or absence of the specific sequence polymorphisms.

48. The method of claim 43, wherein the at least one oligonucleotide primer sets are adapted to identify a specific allele of the HLA-A, HLA-B and HLA-DR genes.

49. The method of claim 43, wherein the at least one oligonucleotide primer sets are adapted to identify a specific allele of the HLA-A, HLA-B, HLA-C, HLA-DR and HLA-DQ genes.

50. The method of claim 43, wherein no more than 96 primer sets are required to identify the specific allele.

51. The method of claim 43, wherein between 48 and 96 primer sets are required to identify the specific allele.

52. The method of claim 43, wherein the step of contacting the nucleic acid sample with the at least one primer set and subjecting the nucleic acid sample and at least one primer set to a nucleic acid amplification reaction is performed in an array.

53. An in vitro method for determining the human leukocyte antigen (HLA) genotype of a nucleic acid sample that has been obtained from a biological sample, comprising the steps of:
(i) providing at least one oligonucleotide primer set;
(ii) contacting the nucleic acid sample with each of the primer sets and subjecting the nucleic acid sample and each primer set to a nucleic acid amplification reaction;
(iii) separating any nucleic acid amplification products produced in each of the nucleic acid amplification reactions using a capillary electrophoresis (CE) separation technique;
(iv) determining the size of the amplification products that have been separated using CE, and correlating the presence and/or absence of specific amplification products with the presence and/or absence of specific sequence polymorphisms associated with HLA alleles in the nucleic acid sample; and
(v) assigning an HLA genotype on the basis of the information derived from the presence and/or absence of the specific sequence polymorphisms associated with HLA alleles in the nucleic acid sample;
wherein each primer set comprises at least one sequence-specific forward primer and at least one sequence-specific reverse primer and is adapted to amplify, in a nucleic acid amplification reaction, two or more target sequences that may be present in the nucleic acid sample, and wherein at least one of the target sequences comprises a specific sequence polymorphism that is known to be associated with an HLA allele, and which may be present in the nucleic acid sample to be genotyped;
and wherein steps (iv) and (v) are carried out using an auto-interpretation software program run on a computer, which auto-interpretation software program avoids the requirement for manual interpretation of data to assign an HLA genotype.

54. The method of claim 53, wherein the software provides an output that reports the genotype information derived on the basis of the presence and/or absence of the specific sequence polymorphisms.

55. The method of claim 53, wherein the at least one oligonucleotide primer sets are adapted to identify a specific allele of the HLA-A, HLA-B and HLA-DR genes.

56. The method of claim 53, wherein the at least one oligonucleotide primer sets are adapted to identify a specific allele of the HLA-A, HLA-B, HLA-C, HLA-DR and HLA-DQ genes.

57. The method of claim 53, wherein no more than 96 primer sets are required to identify the specific allele.

58. The method of claim 53, wherein between 48 and 96 primer sets are required to identify the specific allele.

59. The method of claim 53, wherein the step of contacting the nucleic acid sample with each of the primer sets and subjecting the nucleic acid sample and each primer set to a nucleic acid amplification reaction is performed in an array.

60. A software program for assigning a human leukocyte antigen (HLA) genotype of a nucleic acid sample, which software program:
(i) correlates the presence and/or absence of specific nucleic acid amplification products of expected size with the presence and/or absence of specific sequence polymorphisms in an HLA gene or genes using a means of data comparison; and
(ii) assigns an HLA genotype on the basis of the information derived from the presence and/or absence of the specific sequence polymorphisms associated with HLA alleles in the nucleic acid sample.

61. The software program of claim 60, wherein the means of data comparison in step (i) is one or more look-up table.

62. The software program of claim 60, wherein the means of data comparison in step (i) is one or more look-up table, and wherein a separate look-up table is used to assign an HLA genotype for each HLA gene.

63. The software program of claim 60, wherein the genotype of the HLA-A, HLA-B and HLA-DR genes are assigned.

64. The software program of claim 60, wherein the genotype of the HLA-A, HLA-B, HLA-C, HLA-DR and HLA-DQ genes are assigned.

65. A kit for determining the human leukocyte antigen (HLA) genotype of a nucleic acid sample obtained from a biological sample comprising:
at least one oligonucleotide primer set; and
operating instructions in the form of a protocol for performing the genotyping method;
wherein at least one of the at least one primer sets is a multi-specific primer set comprising at least one sequence-specific forward primer and at least one sequence-specific reverse primer and being adapted to amplify, in a nucleic acid amplification reaction, two or more specific target sequences that may be present in the nucleic acid sample, and wherein each of the specific target sequences comprises a sequence polymorphism that is known to be associated with an HLA allele and which may be present in the nucleic acid sample to be genotyped.

66. The kit of claim 65, wherein the at least one oligonucleotide primer sets are arranged in an array.

67. The kit of claim 65, which further comprises at least one compartment for separately compartmentalising each of the at least one primer sets, and wherein each primer set is pre-aliquoted into a separate one of the compartments.

68. The kit of claim 65, wherein each of the at least one primer sets is pre-aliquoted into a separate well of a 96-well plate.

69. The kit of claim 65, wherein each of the at least one primer sets is pre-aliquoted into a separate well of a 384-well plate.

70. The kit of claim 65, wherein each of the at least one primer sets is dried, preferably freeze dried or lyophilised.

71. The kit of claim 65, wherein each of the multi-specific primer sets comprises two specific primer pairs, each specific primer pair comprising a forward primer and a reverse primer and being adapted to amplify at least a specific target sequence that may be present in the nucleic acid sample.

72. The kit of claim 65, wherein at least one multi-specific primer set comprises two specific primer pairs, each specific primer pair being adapted to amplify a specific target sequence that may be present in the nucleic acid sample, and wherein each of the specific target sequences is in a different genetic locus of the nucleic acid sample.

73. The kit of claim 65, wherein at least one multi-specific primer set comprises two specific primer pairs, each specific primer pair being adapted to amplify a specific target sequence that may be present in the nucleic acid sample, and wherein each of the specific target sequences the same genetic locus of the nucleic acid sample.

74. The kit of claim 65, wherein at least one multi-specific primer set comprises two specific primer pairs, each specific primer pair being adapted to amplify a specific target sequence that may be present in the nucleic acid sample, and wherein each of the specific target sequences the same genetic locus of the nucleic acid sample, and wherein the target sequences overlap.

75. The kit of claim 65, wherein the sequence polymorphism is selected from the group comprising: single nucleotide polymorphisms (SNPs); insertions, substitutions and deletions of one or more nucleotides; and repetitive sequences (for example, microsatellites or repeats).

76. The kit of claim 65, wherein each of the primer sets is adapted to identify a specific allele of the HLA-A, HLA-B or HLA-DR genes.

77. The kit of claim 65, wherein each of the primer sets is adapted to identify a specific allele of the HLA-A, HLA-B, HLA-C, HLA-DR or HLA-DQ genes.

78. The kit of claim 65, wherein no more than 96 primer sets are provided.

79. The kit of claim 79, wherein between 48 and 96 primer sets are provided.

80. The kit of claim 65, wherein at least one primer set comprises one or more primers selected from the group comprising SEQ ID NOS. 1 to 18.

81. The kit of claim 65, which further comprises the software program of claim 60.


SEQUENCE LISTING (18 sequences)

SEQ ID NO: 1 - DNA, 18 bp, Artificial
  Primer sequence
ttgggaccggaacacacg

SEQ ID NO: 2 - DNA, 23 bp, Artificial
  Primer sequence
ctcgctctggttgtagtagcgga

SEQ ID NO: 3 - DNA, 20 bp, Artificial
  Primer sequence
cgggtaccagcaggaacgct

SEQ ID NO: 4 - DNA, 17 bp, Artificial
  Primer sequence
ggtaccagcgggacgct

SEQ ID NO: 5 - DNA, 18 bp, Artificial
  Primer sequence
ggagccactccacgcacc

SEQ ID NO: 6 - DNA, 18 bp, Artificial
  Primer sequence
ggagccactccacgcaca

SEQ ID NO: 7 - DNA, 22 bp, Artificial
  Primer sequence
cccactccatgaggtatttcta

SEQ ID NO: 8 - DNA, 21 bp, Artificial
  Primer sequence
tccactcggtcagtctgtgac

SEQ ID NO: 9 - DNA, 21 bp, Artificial
  Primer sequence
tccactcggtgagtctgtgac

SEQ ID NO: 10 - DNA, 22 bp, Artificial
  Primer sequence
gtcctctcggtgagtctgtgac

SEQ ID NO: 11 - DNA, 20 bp, Artificial
  Primer sequence
tgcatctggacatgcttgct

SEQ ID NO: 12 - DNA, 20 bp, Artificial
  Primer sequence
tggctggaggagactccaaa

SEQ ID NO: 13 - DNA, 24 bp, Artificial
  Primer sequence
acgtttcttggagcaggttaaaca

SEQ ID NO: 14 - DNA, 22 bp, Artificial
  Primer sequence
cgctgcactgtgaagctctcac

SEQ ID NO: 15 - DNA, 22 bp, Artificial
  Primer sequence
cgctgcactgtgaagctctcca

SEQ ID NO: 16 - DNA, 21 bp, Artificial
  Primer sequence
gagtacgcgcgctacaacagt

SEQ ID NO: 17 - DNA, 25 bp, Artificial
  Primer sequence
ctaaggtgactgtgtatccttcaaa

SEQ ID NO: 18 - DNA, 20 bp, Artificial
  Primer sequence
gccttctctcttcctggctg